SEQUENCE LISTING (8 sequences)

SEQ ID NO: 1 - DNA, 20 bp, Artificial Sequence
  Description of Artificial Sequence Synthetic primer
accacagtccatgccatcac

SEQ ID NO: 2 - DNA, 20 bp, Artificial Sequence
  Description of Artificial Sequence Synthetic primer
tccaccaccctgttgctgta

SEQ ID NO: 3 - DNA, 18 bp, Artificial Sequence
  Description of Artificial Sequence Synthetic primer
aaaatggtcccctcggct

SEQ ID NO: 4 - DNA, 18 bp, Artificial Sequence
  Description of Artificial Sequence Synthetic primer
tctgggctcttcagacca

SEQ ID NO: 5 - DNA, 17 bp, Artificial Sequence
  Description of Artificial Sequence Synthetic primer
tgcgggaccatgaagct

SEQ ID NO: 6 - DNA, 19 bp, Artificial Sequence
  Description of Artificial Sequence Synthetic primer
tctcagtgggaattagtca

SEQ ID NO: 7 - DNA, 18 bp, Artificial Sequence
  Description of Artificial Sequence Synthetic primer
tgtcccctgtcccacgat

SEQ ID NO: 8 - DNA, 18 bp, Artificial Sequence
  Description of Artificial Sequence Synthetic primer
agccttgctctgtgccca

(19) United States
(12) Patent Application Publication
(10) Pub. No.: US 2011/0052570 A1
(43) Pub. Date: Mar. 3, 2011

(54) METHOD TO PROGNOSE RESPONSE TO ANTI-EGFR THERAPEUTICS

(75) Inventors: Michael Klagsbrun, Newton Centre, MA (US); Dhara N. Amin, Brighton, MA (US); Kyoko Hida, Newton, MA (US)

(73) Assignee: CHILDREN'S MEDICAL CENTER CORPORATION, Boston, MA (US)

(21) Appl. No.: 12/091,190

(22) PCT Filed: Oct. 24, 2006
(86) PCT No.: PCT/US06/41250

§ 371 Date: Apr. 23, 2008

Related U.S. Application Data
(60) Provisional application No. 60/730,272, filed on Oct. 26, 2005.

(51) Int. Cl. A61K 39/395; C12Q 1/68; G01N 33/574; C12Q 1/02; C12Q 1/18; C40B 30/00; A61K 31/7088; A61K 31/5377; A61K 31/519; A61K 31/517; A61K 31/4709; C07K 16/30; C07K 16/28; A61P 35/00

(52) U.S. Cl. 424/133.1; 424/142.1; 424/174.1; 435/6; 435/7.1; 435/7.92; 435/29; 435/32; 506/7; 514/44 R; 514/234.5; 514/252.16; 514/266.4; 514/313; 530/389.7; 530/389.8

(57) ABSTRACT

The present invention provides methods to determine the likelihood of effectiveness of an EGFR targeting treatment in a subject affected with a tumor based on the expression of EGFR of endothelial cells associated with the tumor. The present invention also provides methods of treating a subject affected with, or at risk for developing cancer with an EGFR targeting treatment and methods to screen for an EGFR targeting treatment.

### GOVERNMENT SUPPORT

This invention was supported, in part, by National Institutes of Health (NIH) Grants Nos. CA37392 and CA45548. The government of the United States has certain rights to the invention.

### BACKGROUND OF THE INVENTION

The epidermal growth factor (EGF) family of type I receptor tyrosine kinases consists of four members: EGFR/ErbB1/HER, ErbB2/Neu/HER2, ErbB3/HER3, and ErbB4/HER4 (1, 2). These receptors are activated by ligands belonging to the EGF family of polypeptide growth factors. The ligands can be divided into four groups based on their binding specificity for the four receptors (1, 2). EGF, transforming growth factor-alpha (TGFα) and amphiregulin (AR) bind only to EGFR. Heparin binding-EGF like growth factor (HB-EGF), betacellulin (BTC), and epiregulin (EPR) bind to both EGFR and ErbB4. Neuregulin 1 (NRG) and NRG2 bind to both ErbB3 and ErbB4, whereas NRG3 and NRG4 bind to ErbB4 alone. Ligand binding induces receptor homo- and hetero-dimerization (3). Dimerization of the EGF receptors increases their intrinsic tyrosine kinase activity resulting in receptor auto-phosphorylation. ErbB2 is not known to bind any ligand, but heterodimerizes with the other EGFG receptors, contributing to the autophosphorylation (and activation) of both hetereodimer subunits and delaying the internalization of the heterodimer from the cell surface. The phosphotyrosine sites recruit downstream adaptor and signaling proteins thus initiating signaling cascades including MAPK, PI3K, and PKC in the cytoplasm (3). Signaling initiated by the EGF receptors impacts cell proliferation, differentiation, adhesion, apoptosis, and migration (2).

EGF receptor family members are important in the etiology of numerous tumors including those of the breast, ovary, lung, colon, nervous system, head and neck, prostate, and pancreas (2). Amplification of EGFR and ERBB2, and subsequent over expression of the protein, is observed in 20-30% of breast tumors and is associated with poor patient prognosis (4). In contrast, melanomas express low to intermediate levels of EGFR (5). The EGF receptor family members, especially EGFR and ErbB2, are targets of anti-cancer therapeutics and some are already FDA approved and in the clinic (6,7). HERCEPTIN® (Trastuzumab, Genentech) is a monoclonal antibody against ErbB2 administered to advanced stage breast cancer patients, while ERBITUX® (Cetuximab, ImClone), a monoclonal antibody against EGFR, is approved for treatment of colon carcinomas. IRESSA® (Gefitinib, AstraZeneca) and TARCEVA® (Erlonitib, Genentech) are small molecule kinase inhibitors of EGFR that are approved for treating non-small cell lung cancer (NSCLC) patients.

There is a need for new treatments for subjects with cancer.

### SUMMARY OF THE INVENTION

We have surprisingly found that EGFR is differentially expressed in tumor endothelial cells relative to normal endothelial cells. Thus, even when the tumor does not express EGFR, EGFR targeting treatments may still be effective. Additionally, by targeting only tumor associated endothelial cells, normal endothelial cells would be unharmed. The present invention provides methods to prognose a subject's response to EGFR and ErbB2 targeting treatments based on the expression status of the subject's tumor associated endothelial cells.

The present invention provides a method for determining the likelihood of effectiveness of an EGFR targeting treatment in a subject affected with a tumor comprising: detecting the presence or absence of EGFR expression in endothelial cells associated with the tumor, wherein the presence of EGFR expression indicates that the EGFR targeting treatment is likely to be effective. In one embodiment, the tumor does not express EGFR.

In one embodiment, EGFR expression is evaluated in a biological sample from said subject. In one embodiment, the biological sample comprises a tumor. In one embodiment, the biological sample comprises tumor endothelial cells.

In one embodiment, the subject's tumor is gastrointestinal cancer, prostate cancer, ovarian cancer, breast cancer, head and neck cancer, lung cancer, non-small cell lung cancer, cancer of the nervous system, kidney cancer, retinal cancer, skin cancer, stomach cancer, liver cancer, pancreatic cancer, genital-urinary cancer, prostate cancer, colorectal cancer, bladder cancer or other cancer.

The present invention provides a method of treating a subject affected with or at risk for developing cancer, comprising detecting the presence or absence of EGFR expression in endothelial cells associated with a tumor in said subject, wherein the subject is administered an EGFR targeting treatment if the presence of the said EGFR expression is detected. In one embodiment, the tumor does not express EGFR.

The present invention provides a method to direct treatment of a subject with a tumor, wherein the EGFR expression status of the endothelial cells associated with the tumor is utilized for the direction of treatment, wherein positive EGFR expression status directs treatment of the subject towards administration of an EGFR targeting treatment.

The present invention provides a kit for detecting the presence or absence of EGFR expression comprising: antibody to EGFR, antibody to tumor endothelial cell antigens, antibody to endothelial cell antigens or any combination thereof.

The present invention provides a method of screening for EGFR targeting agents, wherein an agent is screened for effectiveness targeting EGFR, wherein tumor epithelial cells expressing EGFR are administered the agent, wherein the response of the tumor epithelial cells to the agent is monitored.

In one embodiment, cessation of growth or death of the tumor endothelial cell indicates that the EGFR targeting agent is effective.

In one embodiment, reduction in EGFR phosphorylation indicates that the EGFR targeting agent is effective.

In one embodiment, reduction in EGFR expression indicates that the EGFR targeting agent is effective.

The present invention provides a method of screening for EGFR targeting agents, wherein an agent is screened for effectiveness targeting ErbB2, wherein tumor epithelial cells expressing ErbB2 are administered the agent, wherein the response of the tumor epithelial cells to the agent is monitored.

In one embodiment, cessation of growth or death of the tumor endothelial cell indicates that the ErbB2 targeting agent is effective.

In one embodiment, reduction in ErbB2 phosphorylation indicates that the ErbB2 targeting agent is effective.

In one embodiment, reduction in ErbB2 expression indicates that the ErbB2 targeting agent is effective.

### BRIEF DESCRIPTION OF THE DRAWINGS

FIGS. 1A-1D show comparative expression of the EGF receptor family in tumor and skin EC. FIG. 1A shows A375SM tumor EC and mouse skin EC analyzed by western blotting for the expression of the four EGF receptor family members. FIG. 1B shows western blotting for EGFR expression in A375SM tumor EC (lane 3) and non-tumor derived normal EC including skin-, adipose-, MS1-(transformed mouse EC), HUV- and HMV-EC (lanes 4-8). EGFR expression was also analyzed in A375SM melanoma (lane 1) and MDA-MB-231 breast carcinoma (lane 2) tumor cells. β-Actin blotting was performed as a loading control. FIG. 1C shows FACS analysis of EGFR (black) in tumor EC is shown compared to rabbit IgG control (grey). FIG. 1D shows FACS analysis of ErbB3 (black) in skin EC was carried out with rabbit IgG (grey) used as a control.

FIGS. 2A-2D show activation of EGFR and ErbB2 in tumor EC. FIG. 2A shows serum starved melanoma (tumor EC) and skin EC stimulated with (+) or without (−) EGF. EGFR and ErbB2 phosphorylation (p-EGFR and pErbB2) was detected by immunoprecipitation of the receptors followed western blotting with anti-phosphotyrosine antibody. The same lysates were analysed by western blotting for p-Erk1/2 and blots were stripped and re-probed for Erk1 as a loading control. FIG. 2B shows liposarcoma EC (Liposarc EC) and MDA-MB-231 breast carcinoma EC (BrCa EC) treated with or without EGF. EGFR, ErbB2, and Erk1/2 phosphorylation was detected as in panel A. FIG. 2C shows serum starved A375SM tumor EC (lanes 1-5) and skin EC (lanes 6-10) stimulated with 100 ng/ml EGF, TGFα, HB-EGF, BTC, NRG1β or vehicle (mock) as indicated. EGFR activation was determined by immunoprecipitation with EGFR antibody followed by western blotting with phosphotyrosine antibody. FIG. 2D shows RT-PCR was performed to detect EGF, TGFα, and HB-EGF transcripts in A375SM (melanoma) and MDA-MB-231 (breast cancer) tumor cells. GAPDH was amplified as a control.

FIGS. 3A-3C shows proliferation of tumor EC in response to EGF and inhibition by the AG1478 EGFR kinase inhibitor. FIG. 3A shows the MTT assay to detect proliferating cells in response to EGF in tumor (black) and skin EC (grey) was performed. The absorbance by EGF-treated to non-EGF treated cells is shown as percentage proliferation. Error bars represent standard error of the mean (S.E.M). FIG. 3B shows serum starved cells were pretreated with the indicated concentrations of AG1478 followed by EGF (100 ng/ml) treatment. EGFR phosphorylation was analyzed by western blotting with phospho1068-EGFR antibody. Blots were stripped and re-probed with EGFR to show equal loading. FIG. 3C shows the MTT assay was performed to detect proliferating cells in response to increasing concentration of EGF in absence or presence of 1 μM AG1478. Cell proliferation was analyzed by MTT as in panel A. Tumor EC (black solid), tumor EC with AG1478 (black dotted), skin EC (gray).

FIGS. 4A-4B shows that neuregulin activates ErbB3 receptors on Skin EC inhibiting their proliferation. FIG. 4A shows A375SM tumor EC and skin EC were serum-starved and stimulated with or without NRG1β. Activated ErbB3 (p-ErbB3) was detected by immunoprecipitation with anti-ErbB3 antibody followed by phosphotyrosine western blotting. The blots were stripped and re-probed for ErbB3. The same lysates were analyzed by blotting for p-Erk1/2 and blots were stripped and re-probed for Erk1 as a loading control. FIG. 5B shows cell proliferation measured using the MTT assay. Serum starved cells were treated with increasing doses of NRG1β. The absorbance of NRG-treated compared to vehicle treated cells is shown as percentage proliferation. Tumor EC (black), skin EC (gray). Error bars represent S.E.M from triplicate wells.

FIG. 5 shows that geftinib is effective against EGFR-negative tumors in mice. Tumor volumes were measured every 4 days. Shown is the tumor volume from the start of treatment (day 0) until the end of treatment (day 28) for the Gefitinib (black, n=11) and Control groups (gray, n=11). The error bars represent standard error of the mean. *, P<0.05, two-tailed Student's t test.

FIG. 6 shows Iressa inhibits EGFR negative A375SM melanoma xenograph growth by targeting the EC. FIG. 6A is a RT-PCR for EGFR of A375SM melanoma EC to show melanoma xenoplants do not express human EGFR mRNA, where MDA-MB231 is used as a positive control. FIG. 6B is a RT-PCR using mouse specific EGFR primers to detect EGFR in CD31-positive and CD31-negative fractions of the tumor. FIG. 6C shows a western blot for phosphor-EGFR, phosphor-Erb32, phospho-AKT and AKT of different cells treated with or without Iressa, showing Iressa inhibits EGF induced phosphorylation of EGFR and Erb2 and AKT. FIG. 6D shows a dose-dependent inhibition by Iressa of EGF-dependent cell proliferation of melanoma EC cells, but not normal EC or A375S cells. FIG. 6D shows the reduction of tumor growth on mice bearing A375SM xenographs after 4 weeks of daily administration of Iressa. The error bars represent standard error of the mean. *, P<0.05, two-tailed Student's t test.

### DETAILED DESCRIPTION OF THE INVENTION

The present invention is based on the surprising discovery that in the absence of EGFR expression in a tumor, tumor associated endothelial cells may express EGFR. The present invention provides methods to prognose a subject's response to EGFR targeting treatment based on the expression status of tumor associated endothelial cells. Whereas in normal endothelial cells which express ErbB3 and repress the expression of EGFR, it has surprisingly been discovered that tumor endothelial cells may express EGFR and repress ErbB3. Therefore, it is our discovery that it is possible to target tumor endothelial cells while sparing normal endothelial by the use of anti-EGFR therapeutic intervention.

Definitions

As used herein, “tumor associated endothelial cells” or “tumor endothelial cells” are used interchangeably herein and refer to endothelial cells that comprise the vasculature that supports a tumor. Tumor associated endothelial cells may be found in blood vessels that invade a tumor or that are co-opted by the tumor. Tumor vasculature may be organized abnormally and chaotically compared to the hierarchal branching typical of normal vasculature (reviewed in Hida and Klagsburn. Cancer Res 2005; 65:2507-2510). Gene expression markers may be used to identify tumor endothelial cells and can be found in St Croix et al. Science, 2000; 289:1197-1202; Carson-Walter et al. Cancer Res, 2001; 61:6649-6655; and Hardwick et al. Mol Cancer Ther. 2005; 4(3);413-25, herein incorporated in their entirety. Useful tumor endothelial cell markers include the TEM genes, which are associated with the tumor endothelial cell surface (Carson-Walter et al. supra).

As used herein, “EGFR”, “epidermal growth factor”, “ErbB1”, “HER” or “HER-1”, are used interchangeably herein and refer to native sequence EGFR as disclosed, for example, in Carpenter et al. Ann. Rev. Biochem. 56:881-914 (1987), including variants thereof, e.g. a deletion mutant EGFR as in Humphrey et al. PNAS (USA) 87:4207-4211 (1990). ErbB1 refers to the gene encoding the EGFR protein product.

As used herein, “ErbB-2”, “HER-2”, “Neu”, or “neu proto-oncogene”, encodes a p185 tumor antigen which is a growth factor receptor having extracellular, transmembrane, and intracellular domains. The oncogenic form of this protein (sometimes referred to as “oncogenic ErbB-2” or “c-ErbB-2”) contains a single amino acid point mutation located in the transmembrane domain, causing the receptor to become constitutively active, i.e., active in the absence of ligand. Over-expression of the normal receptor in a cell also causes the cell to become transformed.

An “EGFR associated disease” refers to a disease which is caused by or contributed to by excessive or insufficient EGFR, resulting, e.g., from over-expression of EGFR or a mutation in EGFR or the presence of excess ligand for the receptor. An “EGFR associated cancer” refers to a cancer which is caused by or contributed to by excessive EGFR stimulation, resulting, e.g., from over-expression of EGFR or a mutation in EGFR or the presence of excess ligand for the receptor. Exemplary EGFR associated cancers include carcinomas, e.g., breast carcinoma.

The term “EGFR inhibitor” as used herein refers to a molecule having the ability to inhibit a biological function of a native epidermal growth factor receptor (EGFR), including mutant EGFR. Accordingly, the term “inhibitor” is defined in the context of the biological role of EGFR. While in some embodiments, inhibitors herein specifically interact with, e.g. bind to, an EGFR, molecules that inhibit an EGFR biological activity by interacting with other members of the EGFR signal transduction pathway are also specifically included within this definition. In another embodiment, EGFR biological activity inhibited by an EGFR inhibitor is associated with the development, growth, or spread of a tumor or associated with the development or proliferation, of tumor associated endothelial cells. EGFR inhibitors, without limitation, include peptides, non-peptide small molecules, antibodies, antibody fragments, antisense molecules, and oligonucleotide decoys.

As used herein, the term “prognosis” is used to refer to the prediction of the probable response of a subject to a course of treatment. For example, the methods of the present invention provide for the detection of EGFR expression by tumor associated endothelial cells. A tumor associated endothelial cell that expresses EGFR, even in the absence of EGFR expression by the tumor itself, indicates that therapeutics targeting EGFR, e.g. EGFR inhibitors, will be efficacious in treating the tumor. Thus, the prognosis for a subject includes a prediction of the response of the subject to agents targeting EGFR.

As used herein, the term “subject” or “patient” refers to any mammal. The subject cab be human, but can also be a mammal in need of veterinary treatment, e.g. domestic animals, farm animals, and laboratory animals. For example, the subject may be a subject diagnosed with a benign or malignant tumor, a cancer or a hyperplasia. The subject may be a cancer patient who is-receiving treatment modalities against cancer or has undergone a regimen of treatment, e.g., chemotherapy, radiation and/or surgery. The subject may be a cancer patient whose cancer appears to be regressing.

As used herein, the phrase “gene expression” is used to refer to the transcription of a gene product into mRNA and is also used to refer to the expression of the protein encoded by the gene.

Standard molecular biology techniques known in the art and not specifically described are generally followed as in Sambrook et al., Molecular Cloning: A Laboratory Manual, Cold Springs Harbor Laboratory, New York (1989, 1992), and in Ausubel et al., Current Protocols in Molecular Biology, John Wiley and Sons, Baltimore, Md. (1989). Additionally, standard methods in immunology known in the art and not specifically described are generally followed as in Stites et al. (eds), Basic and Clinical Immunology (8^{th} Edition), Appleton & Lange, Norwalk, Conn. (1994) and Mishell and Shiigi (eds), Selected Methods in Cellular Immunology, W. H. Freeman and Co., New York (1980).

### Methods to Detect EGFR

The present invention also provides, in other aspects, methods for detecting EGFR expression in tumor associated endothelial cells by measuring EGFR expression in samples taken from a tumor biopsy or biopsy comprising tumor associated endothelial cells, and determining whether EGFR is expressed in the tumor associated endothelial cells. The various techniques, including hybridization based and amplification based methods, for measuring and evaluating EGFR expression are provided herein and known to those of skill in the art. The invention thus provides methods for detecting EGFR expression at the RNA or protein levels wherein both results are indicative of a subject's response to anti-EGFR therapeutics.

Expression is detected in a biological sample obtained from the subject. The biological sample may be a tumor biopsy or biopsy comprising tumor endothelial cells. The biological sample may be cells obtained from a blood sample from the subject. The biological sample may be obtained during surgical resection of a tumor. RNA, either total RNA or mRNA, may be isolated or extracted from the biological sample. Protein may be extracted from the biological sample. Extracted or isolated nucleic acid or protein material may be used to detection of gene expression. In one preferred embodiment, expression of EGFR is detected in the biological sample, e.g. a biopsy, itself.

In another preferred embodiment, blood is collected from the subject and nucleated cells isolated from the blood, e.g. by ficoll gradient and cytospin tube use. Nucleated cells may include tumor endothelial cells and endothelial cells. Tumor endothelial cells, and optionally endothelial cells, may be detected by immunohistochemistry or RNA-FISH, as described below, using antibodies to tumor endothelial cell markers, antibodies to endothelial cell markers or FISH probes to tumor endothelial cell markers, FISH probes to endothelial cell markers. In another preferred embodiment, the tumor endothelial cells, and optionally endothelial cells, are detected and/or isolated by flow cytometric means, as described below. Kits, reagents and equipment by Immunicon Corporation (Huntingdon Valley, Pa.) may be useful in detection and/or isolation of circulating endothelial cells and/or tumor endothelial cells.

The methods of the present invention are particularly applicable to subjects whose tumors do not express EGFR or EGFR variants, thus determination of EGFR expression or presence of an EGFR variant may be determined prior to, concurrent with or subsequent to determination of EGFR expression by the tumor endothelial cells. Determination of EGFR expression in the tumor may be determined by any of the methods described below, or any other method known in the art, for detection of EGFR expression in endothelial cells. The same methods or different methods may be utilized for detecting gene expression in the tumor as for detecting expression in the tumor endothelial cells.

The present invention encompasses methods of detecting gene expression known to those of skill in the art, see, for example, Boxer, J. Clin. Pathol. 53:19-21(2000). Such techniques include in situ hybridization (Stoler, Clin. Lab. Med. 12:215-36 (1990), using radioisotope or fluorophore-labeled probes; reverse transcription and polymerase chain reaction (RT-PCR); Northern blotting, dot blotting and other techniques for detecting individual genes. The probes or primers selected for gene expression evaluation are highly specific to avoid detecting closely related homologous genes. Alternatively, antibodies may be employed that recognize EGFR antigens in various immunological assays, including immunohistochemical, western blotting, ELISA assays, etc.

In another embodiment, the methods further involve obtaining a control biological sample and detecting EGFR expression in this control sample, such that the presence or absence of EGFR expression in the control sample is determined. A negative control sample is useful if there is an absence of EGFR expression, whereas a positive control sample is useful if there is a presence of EGFR expression. For the negative control, the sample may be from the same subject as the test sample (i.e. different location such as tumor associated endothelial cells versus non-tumor associated endothelial cells) or may be from a different subject known to have an absence of EGFR expression.

In one preferred embodiment, techniques that provide histological information about the biological sample are used, for example immunohistochemical or FISH-based techniques. Histological information may be used to determine that the cells expressing EGFR are tumor endothelial cells. Immunohistochemical or FISH-based techniques may also be used to identify cells that express endothelial markers and/or tumor endothelial markers, as well as to identify cells that express EGFR.

Any of the gene expression detection methods useful in the methods of the present invention may be used to detect expression of endothelial cell markers or tumor endothelial cell markers e.g. PECAM, (CD31), VEGFR-1, VEGFR-2, neuropilin (NRP) 1, NRP2, TIE1, TIE2, VE-cadherin (CDH5) or TEMs or other tumor endothelial marker (Science, 2000; 289:1197-1202; Cancer Res, 2001; 61:6649-6655; Mol Cancer Ther. 2005; 4(3):413-25), in the biological sample in order to determine whether the biological sample contains tumor endothelial cells.

Any of the following gene transcription and polypeptide or protein expression assays can be used to detect mRNA transcription and/or protein expression for EGFR, endothelial cell marker(s), tumor endothelial cell marker(s) or any combination thereof.

Amplification-Based Assays

In one embodiment, amplification-based assays can be used to detect, and optionally quantify, EGFR expression. In such amplification-based assays, the EGFR mRNA in the sample obtained from the subject act as template(s).in an amplification reaction carried out with a nucleic acid primer that contains a detectable label or component of a labeling system. Suitable amplification methods include, but are not limited to, polymerase chain reaction (PCR); reverse-transcription PCR (RT-PCR); ligase chain reaction (LCR) (see Wu and Wallace (1989) Genomics 4: 560, Landegren et al. (1988) Science 241: 1077, and Barringer et al. (1990) Gene 89: 117; transcription amplification (Kwoh et al. (1989) Proc. Natl. Acad. Sci. USA 86: 1173), self-sustained sequence replication (Guatelli et al. (1990) Proc. Nat. Acad. Sci. USA 87: 1874); dot PCR, and linker adapter PCR, etc. The known nucleic acid sequence for EGFR (Accession No.: NM_{—}005228, NM_{—}201282, NM_{—}201283, NM_{—}201284) is sufficient to enable one of skill to routinely select primers to amplify any portion of the gene.

PCR-Based Gene Expression Detection Methods

Reverse Transcriptase PCR (RT-PCR)

One of the most sensitive and most flexible PCR-based gene expression detection methods is RT-PCR, which can be used to determine presence or absence of expression and also to quantitate levels of gene expression.

The first step is the isolation of mRNA from a target sample. The starting material is typically total RNA isolated from human tumors, and may also include corresponding normal tissues. Thus RNA can be isolated from a variety of primary tumors, including breast, lung, colon, prostate, brain, liver, kidney, pancreas, spleen, thymus, testis, ovary, uterus, head and neck, etc., tumor. mRNA can be extracted, for example, from frozen or archived paraffin-embedded and fixed, e.g. formalin-fixed, tissue samples.

General methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest. 56:A67 (1987), and De Andrs et al., BioTechniques 18:42044 (1995). In particular, RNA isolation can be performed using purification kit, buffer set and protease from commercial manufacturers, such as Qiagen (Valencia, Calif.), according to the manufacturer's instructions. For example, total RNA from cells in culture can be isolated using Qiagen RNeasy mini-columns. Other commercially available RNA isolation kits include MasterPure™ Complete DNA and RNA Purification Kit (EPICENTRE®, Madison, Wis.), and Paraffin Block RNA Isolation Kit (Ambion, Inc., Austin, Tex.). Total RNA from tissue samples can be isolated using RNA Stat-60 (Tel-Test, Friendswood, Tex.). RNA prepared from tumor can be isolated, for example, by cesium chloride density gradient centrifugation.

As RNA cannot serve as a template for PCR, the first step in gene expression detection by RT-PCR is the reverse transcription of the RNA template into cDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction. Methods for reverse transcription of template RNA to cDNA are well known to persons skilled in the art, and are encompassed in the methods of this invention.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TaqMan® PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TaqMan® RT-PCR can be performed using commercially available equipment, such as, for example, ABI PRISM 7700™ Sequence Detection System™ (Perkin-Elmer-Applied Biosystems, Foster City, Calif, USA), or Lightcycler (Roche Molecular Biochemicals, Mannheim, Germany). In a preferred embodiment, the 5′ nuclease procedure is run on a real-time quantitative PCR device such as the ABI PRISM 7700™ Sequence Detection System™. The system consists of a thermocycler, laser, charge-coupled device (CCD), camera and computer. The system amplifies samples in a 96-well format on a thennocycler. During amplification, laser-induced fluorescent signal is collected in real-time through fiber optics cables for all 96 wells, and detected at the CCD. The system includes software for running the instrument and for analyzing the data.

5′-Nuclease assay data are initially expressed as Ct, or the threshold cycle. As discussed above, fluorescence values are recorded during every cycle and represent the amount of product amplified to that point in the amplification reaction. The point when the fluorescent signal is first recorded as statistically significant is the threshold cycle (Ct).

To minimize errors and the effect of sample-to-sample variation, RT-PCR is usually performed using an internal standard. The ideal internal standard is expressed at a relatively constant level among different tissues, and is unaffected by the experimental treatment. RNAs frequently used to normalize patterns of gene expression are mRNAs for the housekeeping genes glyceraldehyde-3-phosphate-dehydrogenase (GAPDH) and β-actin.

A more recent variation of the RT-PCR technique is the real time quantitative PCR, which measures PCR product accumulation through a dual-labeled fluorigenic probe (i.e., TaqMan® probe). Real time PCR is compatible both with quantitative competitive PCR, where internal competitor for each target sequence is used for normalization, and with quantitative comparative PCR using a normalization gene contained within the sample, or a housekeeping gene for RT-PCR. For further details see, e.g. Held et al., Genome Research 6:986-994 (1996).

Real-time PCR can be performed, for example, using a Perkin Elmer/Applied Biosystems (Foster City, Calif.) 7700 Prism instrument. Matching primers and fluorescent probes can be designed for genes of interest using, for example, the primer express program provided by Perkin Elmer/Applied BioSystems (Foster City, Calif.). Optimal concentrations of primers and probes can be initially determined by those of ordinary skill in the art, and control (for example, beta-actin) primers and probes may be obtained commercially from, for example, Perkin Elmer/Applied Biosystems (Foster City, Calif.). To quantitate the amount of the specific nucleic acid of interest in a sample, a standard curve is generated using a control. Standard curves may be generated using the Ct values determined in the real-time PCR, which are related to the initial concentration of the nucleic acid of interest used in the assay. Standard dilutions ranging from 10-10⁶ copies of the gene of interest are generally sufficient. In addition, a standard curve is generated for the control sequence. This permits standardization of initial content of the nucleic acid of interest in a tissue sample to the amount of control for comparison purposes.

Methods of real-time quantitative PCR using TaqMan probes are well known in the art. Detailed protocols for real-time quantitative PCR are provided, for example, for RNA in: Gibson et al., 1996, A novel method for real time quantitative RT-PCR. Genome Res., 10:995-1001; and for DNA in: Heid et al., 1996, Real time quantitative PCR. Genome Res., 10:986-994.

MassARRAY System

In the MassARRAY-based gene expression profiling method, developed by Sequenom, Inc. (San Diego, Calif.) following the isolation of RNA and reverse transcription, the obtained cDNA is spiked with a synthetic DNA molecule (competitor), which matches the targeted cDNA region in all positions, except a single base, and serves as an internal standard. The cDNA/competitor mixture is PCR amplified and is subjected to a post-PCR shrimp alkaline phosphatase (SAP) enzyme treatment, which results in the dephosphorylation of the remaining nucleotides. After inactivation of the alkaline phosphatase, the PCR products from the competitor and cDNA are subjected to primer extension, which generates distinct mass signals for the competitor- and cDNA-derives PCR products. After purification, these products are dispensed on a chip array, which is pre-loaded with components needed for analysis with matrix-assisted laser desorption ionization time-of-flight mass spectrometry (MALDI-TOF MS) analysis. The cDNA present in the reaction is then quantified by analyzing the ratios of the peak areas in the mass spectrum generated. For further details see, e.g. Ding and Cantor, Proc. Natl. Acad. Sci. USA 100:3059-3064 (2003).

Other PCR-Based Methods

Further PCR-based techniques include, for example, differential display (Liang and Pardee, Science 257:967-971 (1992)); amplified fragment length polymorphism (iAFLP) (Kawamoto et al., Genome Res. 12:1305-1312 (1999)); BeadArray™. technology (Illumina, San Diego, Calif.; Oliphant et al., Discovery of Markers for Disease (Supplement to Biotechniques), June 2002; Ferguson et al., Analytical Chemistry 72:5618 (2000)); BeadsArray for Detection of Gene Expression (BADGE), using the commercially available Luminex100 LabMAP system and multiple color-coded microspheres (Luminex Corp., Austin, Tex.) in a rapid assay for gene expression (Yang et al., Genome Res. 11:1888-1898 (2001)); and high coverage expression profiling (HiCEP) analysis (Fukumura et al., Nucl. Acids. Res. 31(16) e94 (2003)).

Other suitable amplification methods include, but are not limited to ligase chain reaction (LCR) (see Wu and Wallace (1989) Genomics 4:560, Landegren et al. (1988) Science 241:1077, and Barringer et al. (1990) Gene 89:117), transcription amplification (Kwoh et al. (1989) Proc. Natl. Acad. Sci. USA 86:1173), self-sustained sequence replication (Guatelli et al. (1990) Proc. Nat. Acad. Sci. USA 87:1874), dot PCR, and linker adapter PCR, etc.

Hybridization-Based Assays

Hybridization assays can be used to detect EGFR transcription and to detect endothelial cell and/or tumor endothelial cell marker transcription. Hybridization-based assays include, but are not limited to, methods such as Northern blots or RNA in situ hybridization, e.g. fluorescent in situ hybridization (FISH). The methods can be used in a wide variety of formats including, but not limited to substrate, e.g. membrane or glass, bound methods or array-based approaches as described below.

Nucleic acid hybridization simply involves contacting a nucleic acid probe with sample polynucleotides under conditions where the probe and its complementary target nucleotide sequence can form stable hybrid duplexes through complementary base pairing. The nucleic acids that do not form hybrid duplexes are then washed away leaving the hybridized nucleic acids to be detected, typically through detection of an attached detectable label or component of a labeling system. Methods of detecting and/or quantifying polynucleotides using nucleic acid hybridization techniques are known to those of skill in the art (see Sambrook et al. supra). Hybridization techniques are generally described in Hames and Higgins (1985) Nucleic Acid Hybridization, A Practical Approach, IRL Press; Gall and Pardue (1969) Proc. Natl. Acad. Sci. USA 63: 378-383; and John et al. (1969) Nature 223: 582-587. Methods of optimizing hybridization conditions are described, e.g., in Tijssen (1993) Laboratory Techniques in Biochemistry and Molecular Biology, Vol. 24: Hybridization With Nucleic Acid Probes, Elsevier, N.Y.).

The nucleic acid probes used herein for detection of EGFR mRNA can be full-length or less than the full-length of the EGFR transcript. Shorter probes are generally empirically tested for specificity. Preferably, nucleic acid probes are at least about 15, and more preferably about 20 bases or longer, in length. (See Sambrook et al. for methods of selecting nucleic acid probe sequences for use in nucleic acid hybridization.) Visualization of the hybridized probes allows the qualitative determination of the presence or absence of the channel subunit mRNA of interest, and standard methods (such as, e.g., densitometry where the nucleic acid probe is radioactively labeled) can be used to quantify the level of EGFR expression.)

A variety of additional nucleic acid hybridization formats are known to those skilled in the art. Standard formats include sandwich assays and competition or displacement assays. Sandwich assays are commercially useful hybridization assays for detecting or isolating polynucleotides. Such assays utilize a “capture” nucleic acid covalently immobilized to, a solid support and a labeled “signal” nucleic acid in solution. The sample provides the target polynucleotide. The capture nucleic acid and signal nucleic acid each hybridize with the target polynucleotide to form a “sandwich” hybridization complex.

Northern Blot

One method for evaluating EGFR transcription in a sample involves a Northern transfer. Methods for doing Northern Blots are known to those of skill in the art (see Current Protocols in Molecular Biology, Ausubel, et al., Eds., Greene Publishing and Wiley-Interscience, New York, 1995, or Sambrook et al., Molecular Cloning: A Laboratory Manual, 2d Ed. vol. 1-3, Cold Spring Harbor Press, NY, 1989). In such an assay, the total RNA or polyA RNA (typically fragmented and separated on an electrophoretic gel) is hybridized to a probe specific for the target region.

Fluorescence in Situ Hybridization (FISH)

In another embodiment, RNA-FISH is used to determine EGFR transcription in a sample. Fluorescence in situ hybridization (FISH) is known to those of skill in the art (see Angerer, 1987 Meth. Enzymol., 152: 649). Generally, in situ hybridization comprises the following major steps: (1) fixation of tissue or biological structure to be analyzed; (2) prehybridization treatment of the biological structure to increase accessibility of target RNA, and to reduce nonspecific binding; (3) hybridization of the mixture of nucleic acids to the nucleic acid in the biological structure or tissue; (4) post-hybridization washes to remove nucleic acid fragments not bound in the hybridization, and (5) detection of the hybridized nucleic acid fragments.

In a typical in situ hybridization assay, cells or tissue sections are fixed to a solid support, typically a glass slide. If a nucleic acid is to be probed, the cells are typically denatured with heat or alkali. The cells are then contacted with a hybridization solution at a moderate temperature to permit annealing of labeled probes specific to the nucleic acid sequence encoding the protein. The targets, e.g., cells, are then typically washed at a predetermined stringency or at an increasing stringency until an appropriate signal to noise ratio is obtained.

The probes used in such applications are typically labeled, for example, with radioisotopes or fluorescent reporters. Preferred probes are sufficiently long, for example, from about 50, 100, or 200 nucleotides to about 1000 or more nucleotides, to enable specific hybridization with the target nucleic acid(s) under stringent conditions.

In some applications it is necessary to block the hybridization capacity of repetitive sequences. Thus, in some embodiments, tRNA, human genomic DNA, salmon sperm DNA or Cot-1 DNA is used to block non-specific hybridization.

Thus, in one embodiment of the present invention, the presence or absence of EGFR expression is determined by RNA-FISH. In one preferred embodiment, probes directed to endothelial cell markers, e.g. PECAM (CD31), VEGFR-1, VEGFR-2, neuropilin (NRP) 1, NRP2, TIE1, TIE2, VE-cadherin (CDH5) or probes directed to tumor endothelial markers, e.g. TEMS or others (Science, 2000; 289:1197-1202; Cancer Res, 2001; 61:6649-6655; Mol Cancer Ther. 2005 March; 4(3):413-25) are utilized. Colocalization of endothelial cell markers or tumor endothelial cell markers with EGFR markers confirm that the cells expressing EGFR are tumor endothelial cells.

In one embodiment, EGFR expression is determined in cells isolated from blood. Endothelial cell marker expression in combination with EGFR expression may indicate that the cell is a tumor endothelial cell positive for EGFR expression. Tumor endothelial cell marker expression in combination with EGFR expression may indicate that the cell is a tumor endothelial cell positive for EGFR expression.

Microarray Base Expression Analysis

In one embodiment, the methods of the invention can be utilized in array-based hybridization formats for the detection of EGFR in the biological sample. In an array format, a large number of different hybridization reactions can be run essentially “in parallel.” This provides rapid, essentially simultaneous, evaluation of a number of hybridizations in a single experiment. Methods of performing hybridization reactions in array based formats are well known to those of skill in the art (see, e.g., Pastinen (1997) Genome Res. 7: 606-614; Jackson (1996) Nature Biotechnology 14:1685; Chee (1995) Science 274: 610; WO 96/17958, Pinkel et al. (1998) Nature Genetics 20: 207-211).

Arrays, particularly nucleic acid arrays, can be produced according to a wide variety of methods well known to those of skill in the art. For example, in a simple embodiment, “low-density” arrays can simply be produced by spotting (e.g. by hand using a pipette) different nucleic acids at different locations on a solid support (e.g. a glass surface, a membrane, etc.). This simple spotting approach has been automated to produce high-density spotted microarrays. For example, U.S. Pat. No. 5,807,522 describes the use of an automated system that taps a microcapillary against a surface to deposit a small volume of a biological sample. The process is repeated to generate high-density arrays. Arrays can also be produced using oligonucleotide synthesis technology. Thus, for example, U.S. Pat. No. 5,143,854 and PCT Patent Publication Nos. WO 90/15070 and 92/10092 teach the use of light-directed combinatorial synthesis of high-density oligonucleotide microarrays. Synthesis of high-density arrays is also described in U.S. Pat. Nos. 5,744,305; 5,800,992; and 5,445,934.

Hybridization assays according to the invention can also be carried out using a MicroElectroMechanical System (MEMS), such as the Protiveris' multicantilever array.

EGFR mRNA is detected in the above-described polynucleotide-based assays by means of a detectable label. Any of the labels discussed above can be used in the polynucleotide-based assays of the invention. The label may be added to a probe or primer or sample polynucleotides prior to, or after, the hybridization or amplification. So called “direct labels” are detectable labels that are directly attached to or incorporated into the labeled polynucleotide prior to conducting the assay. In contrast, so called “indirect labels” are joined to the hybrid duplex after hybridization. In indirect labeling, one of the polynucleotides in the hybrid duplex carries a component to which the detectable label binds. Thus, for example, a probe or primer can be biotinylated before hybridization. After hybridization, an avidin-conjugated fluorophore can bind the biotin-bearing hybrid duplexes, providing a label that is easily detected. For a detailed review of methods of the labeling and detection of polynucleotides, see Laboratory Techniques in Biochemistry and Molecular Biology, Vol. 24: Hybridization With Nucleic Acid Probes, P. Tijssen, ed. Elsevier, N.Y., (1993)).

In an alternative embodiment of the present invention, EGFR mRNA expression is analyzed via microarray-based platforms. Microarray technology offers high resolution. Details of various microarray methods can be found in the literature. See, for example, U.S. Pat. No. 6,232,068; Pollack et al., Nat. Genet., 23(1):41-6, (1999), Pastinen (1997) Genome Res. 7: 606-614; Jackson (1996) Nature Biotechnology 14:1685; Chee (1995) Science 274: 610; WO 96/17958, Pinkel et al. (1998) Nature Genetics 20: 207-211 and others.

Hybridization protocols suitable for use with the methods of the invention are described, e.g., in Albertson (1984) EMBO J. 3: 1227-1234; Pinkel (1988) Proc. Natl. Acad. Sci. USA 85: 9138-9142; EPO Pub. No. 430,402; Methods in Molecular Biology, Vol. 33: In Situ Hybridization Protocols, Choo, ed., Humana Press, Totowa, N.J. (1994), Pinkel et al. (1998) Nature Genetics 20: 207-211, or of Kallioniemi (1992) Proc. Natl Acad Sci USA 89:5321-5325 (1992), etc.

The sensitivity of the hybridization assays may be enhanced through use of a nucleic acid amplification system that multiplies the target nucleic acid being detected. Examples of such systems include the polymerase chain reaction (PCR) system and the ligase chain reaction (LCR) system. Other methods recently described in the art are the nucleic acid sequence based amplification (NASBAO, Cangene, Mississauga, Ontario) and Q Beta Replicase systems.

The sensitivity of the hybridization assays can be enhanced through use of a polynucleotide amplification system that multiplies the target polynucleotide being detected. Examples of such systems include the polymerase chain reaction (PCR) system and the ligase chain reaction (LCR) system. Other methods recently described in the art are the nucleic acid sequence based amplification (NASBAO, Cangene, Mississauga, Ontario) and Q Beta Replicase systems.

Detection and quantification of gene expression, e.g. EGFR expression, may be carried out through direct hybridization based assays or amplification based assays. The hybridization based techniques for measuring gene transcript are known to those skilled in the art (Sambrook et al., Molecular Cloning: A Laboratory Manual, 2d Ed. vol. 1-3, Cold Spring Harbor Press, NY, 1989). For example, one method for evaluating the presence, absence, or quantity of EGFR-gene expression is by Northern blot. Isolated mRNAs from a given biological subject are electrophoresed to separate the mRNA species, and transferred from the gel to a membrane, for example, a nitrocellulose or nylon filter. Labeled EGFR probes are then hybridized to the membrane to identify and quantify the respective mRNAs. The example of amplification based assays include RT-PCR, which is well known in the art (Ausubel et al., Current Protocols in Molecular Biology, eds. 1995 supplement). In a preferred embodiment, quantitative RT-PCR is used to allow the numerical comparison of the level of respective EGFR mRNAs in different samples. A Real-Time or TaqMan-based assay also can be used to EGFR gene transcription.

Polypeptide-Based Assays

Protein expression, e.g. EGFR expression, can be detected and quantified by any of a number of methods well known to those of skill in the art. Examples of analytic biochemical methods suitable for detecting EGFR protein include electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunohistochemistry, immunocytochemistry, FACS scanning, immunoblotting, immunoprecipitation, affinity chromatography, immunoelectrophoresis, radioimmunoassay (RIA), enzyme-linked immunosorbent assays (ELISAs), immunofluorescent assays, Western blotting, and the like.

Protein expression, e.g. EGFR expression, can be detected and quantified using various well-known immunological assays. Immunological assays refer to assays that utilize an antibody (e.g., polyclonal, monoclonal, chimeric, humanized, scFv, and fragments thereof) that specifically binds to creatine transporter polypeptide (or a fragment thereof). A number of well-established immunological assays suitable for the practice of the present invention are known, and include ELISA, radioimmunoassay (RIA), immunoprecipitation, immunofluorescence, and Western blotting.

Expression of endothelial cell markers or tumor endothelial cell markers, e.g. PECAM, CD31, VEGFR-1, VEGFR-2, neuropilin (NRP) 1, and NRP2 are endothelial cell markers, including tumor endothelial cells, or tumor endothelial markers such as the TEMs (Cancer Res, 2001; 61:6649-6655) and others (Science, 2000; 289:1197-1202; Mol Cancer Ther. 2005; 4(3):413-25), in the biological sample indicate that the biological sample contains endothelial cells.

The EGFR antibodies (preferably anti-mammalian; more preferably anti-human), polyclonal or monoclonal, to be used in the immunological assays of the present invention are commercially available from a variety of commercial suppliers, e.g., AbCam (Cambridge UK and Cambridge, Mass.), Invitrogen Corp. (Carlsbad, Calif.), Bethyl Laboratories (Montgomery, Tex.) and Novus Biologicals (Littleton, Colo.). Alternatively, antibodies may be produced by methods well known to those skilled in the art, e.g., as described in Harlow et al., Antibodies: A Laboratory Manual, 2nd Ed; Cold Spring Harbor Laboratory Press, Cold Spring Harbor, N.Y. (1988). For example, monoclonal antibodies to EGFR, preferably mammalian; more preferably human, can be produced by generation of hybridomas in accordance with known methods. Hybridomas formed in this manner are then screened using standard methods, such as ELISA, to identify one or more hybridomas that produce an antibody that specifically binds to the antigen of interest. Full-length antigen of interest, e.g. EGFR, may be used as the immunogen, or, alternatively, antigenic peptide fragments of the antigen of interest may be used.

As an alternative to preparing monoclonal antibody-secreting hybridomas, a monoclonal antibody to the antigen of interest, e.g. EGFR, may be identified and isolated by screening a recombinant combinatorial immunoglobulin library (e.g., an antibody phage display library) to thereby isolate immunoglobulin library members that bind to the antigen of interest, e.g. EGFR. Kits for generating and screening phage display libraries are commercially available from, e.g., Dyax Corp. (Cambridge, Mass.) and Maxim Biotech (South San Francisco, Calif.). Additionally, examples of methods and reagents particularly amenable for use in generating and screening antibody display libraries can be found in the literature.

Polyclonal sera and antibodies may be produced by immunizing a suitable subject, such as a rabbit, with the antigen of choice, e.g. EGFR, preferably mammalian; more preferably human, or an antigenic fragment thereof. The antibody titer in the immunized subject may be monitored over time by standard techniques, such as with ELISA, using immobilized marker protein. If desired, the antibody molecules directed against the antigen of interest, e.g. EGFR, may be isolated from the subject or culture media and further purified by well-known techniques, such as protein A chromatography, to obtain an IgG fraction.

Fragments of antibodies to the antigen of interest, e.g. EGFR, may be produced by cleavage of the antibodies in accordance with methods well known in the art. For example, immunologically active F(ab′) and F(ab′)₂ fragments may be generated by treating the antibodies with an enzyme such as pepsin. Additionally, chimeric, humanized, and single-chain antibodies to the antigen of interest, comprising both human and nonhuman portions, may be produced using standard recombinant DNA techniques. Humanized antibodies to the antigen of interest may also be produced using transgenic mice that are incapable of expressing endogenous immunoglobulin heavy and light chain genes, but which can express human heavy and light chain genes.

Antibody production is provided by the present invention. Antibodies can be prepared against the immunogen, or any portion thereof, for example a synthetic peptide based on the sequence. As stated above, antibodies are used in assays and are therefore used in determining if the appropriate enzyme has been isolated. Antibodies can also be used for removing enzymes from red cell suspensions after enzymatic conversion. Immunogens can be used to produce antibodies by standard antibody production technology well known to those skilled in the art as described generally in Harlow and Lane, Antibodies: A Laboratory Manual, Cold Springs Harbor Laboratory, Cold Spring Harbor, N.Y., 1988 and Borrebaeck, Antibody Engineering-A Practical Guide, W. H. Freeman and Co., 1992. Antibody fragments can also be prepared from the antibodies and include Fab, F(ab′).sub.2, and Fv by methods known to those skilled in the art.

In the immunological assays of the present invention, the antigen, e.g. EGFR, is typically detected directly (i.e., the antibody to the antigen of interest is labeled) or indirectly (i.e., a secondary antibody that recognizes the antibody to the antigen of interest is labeled) using a detectable label. The particular label or detectable group used in the assay is usually not critical, as long as it does not significantly interfere with the specific binding of the antibodies used in the assay.

The immunological assays of the present invention may be competitive or noncompetitive. In competitive assays, the amount of EGFR in a sample is measured indirectly by measuring the amount of added (exogenous) EGFR displaced from a capture agent, i.e. an anti-EGFR antibody, by the EGFR in the sample. In noncompetitive assays, the amount of EGFR in a sample is directly measured. In a preferred noncompetitive “sandwich” assay, the capture agent (e.g., a first antibody) is bound directly to a solid support (e.g., membrane, microtiter plate, test tube, dipstick, glass or plastic bead) where it is immobilized. The immobilized agent then captures any antigen of interest present in the sample. The immobilized antigen of interest can then be detected using a second labeled antibody to the antigen of interest. Alternatively, the second antibody can be detected using a labeled secondary antibody that recognizes the second antibody.

A preferred method of measuring the expression of the antigen of interest, e.g. EGFR, is by antibody staining with an antibody that binds specifically to the antigen employing a labeling strategy that makes use of luminescence or fluorescence. Such staining may be carried out on fixed tissue or cells that are ultimately viewed and analyzed under a microscope. Staining carried out in this manner can be scored visually or by using optical density measurements. Staining may also be carried out using either live or fixed whole cells in solution, e.g. cells isolated from blood. Such cells can be analyzed using a fluorescence activated cell sorter (FACS), which can determine both the number of cells stained and the intensity of the luminescence or fluorescence. Such techniques are well known in the art, and exemplary techniques are described in Luwor et al. ((2001), Cancer Res. 61:5355-61). One of skill in the art will realize that other techniques of detecting expression might be more or less sensitive than these techniques. As meant herein, cells express little or no antigen if little or no antigen can be detected using an antibody staining technique that relies on luminescence or fluorescence.

Alternatively, EGFR expression in endothelial cells and/or tumors can be detected in vivo in a subject by introducing into the subject a labeled antibody to the EGFR protein. For example, the antibody can be labeled with a radioactive marker whose presence and location in a subject can be detected by standard imaging techniques.

In one embodiment, a EGFR pharmDx™ Kit from DakoCytomation (Glostrup, Denmark) is used to detect EGFR in the sample.

In one preferred embodiment, immunohistochemistry (“IHC”) and immunocytochemistry (“ICC”) techniques, for example, may be used. IHC is the application of immunochemistry to tissue sections, whereas ICC is the application of immunochemistry to cells or tissue imprints after they have undergone specific cytological preparations such as, for example, liquid-based preparations. —Immunochemistry is a—family of techniques based on the use of a specific antibody, wherein antibodies are used to specifically target molecules inside or on the surface of cells. The antibody typically contains a marker that will undergo a biochemical reaction, and thereby experience a change color, upon encountering the targeted molecules. In some instances, signal amplification may be integrated into the particular protocol, wherein a secondary antibody, that includes the marker stain, follows the application of a primary specific antibody.

Immunohistochemical assays are known to those of skill in the art (e.g., see Jalkanen, et al., J. Cell. Biol. 101:976-985 (1985); Jalkanen, et al., J. Cell. Biol. 105:3087-3096 (1987).

Typically, for immunohistochemistry, tissue sections are obtained from a patient and fixed by a suitable fixing agent such as alcohol, acetone, and paraformaldehyde, to which is reacted an antibody. Conventional methods for immunohistochemistry are described in Harlow and Lane (eds) (1988) In “Antibodies A Laboratory Manual”, Cold Spring Harbor Press, Cold Spring Harbor, N.Y.; Ausbel et al (eds) (1987), in Current Protocols In Molecular Biology, John Wiley and Sons (New York, N.Y.). Biological samples appropriate for such detection assays include, but are not limited to, cells, tissue biopsy, whole blood, plasma, serum, sputum, cerebrospinal fluid, breast aspirates, pleural fluid, urine and the like.

For direct labeling techniques, a labeled antibody is utilized. For indirect labeling techniques, the sample is further reacted with a labeled substance.

Alternatively, immunocytochemistry may be utilized. In general, cells are obtained from a patient and fixed by a suitable fixing agent such as alcohol, acetone, and paraformaldehyde, to which is reacted an antibody. Methods of immunocytological staining of human samples is known to those of skill in the art and described, for example, in Brauer et al., 2001 (FASEB J, 15, 2689-2701), Smith-Swintosky et al., 1997.

Colocalization of antibodies to endothelial cell and/or tumor endothelial cell markers with antibodies to EGFR confirm that the cells expressing EGFR are tumor endothelial cells.

Immunological methods of the present invention are advantageous because they require only small quantities of biological material. Such methods may be done at the cellular level and thereby necessitate a minimum of one cell. Preferably, several cells are obtained from a patient affected with or at risk for developing cancer and assayed according to the methods of the present invention.

Endothelial cells circulating in the subject may include circulating endothelial progenitor cells, endothelial cells and tumor endothelial cells. In one embodiment, endothelial cells are collected from the peripheral blood of the subject. Blood may be purified to isolate nucleated cells, e.g. by Ficoll gradient and cytospin tubes. The nucleated cells may be analyzed by immunohistochemistry to determine the presence of tumor endothelial cells. Alternatively, the nucleated cells may be analyzed by antibodies to tumor endothelial cell markers, and optionally by antibodies to endothelial cell markers, e.g. fluorescently-tagged antibodies, antibodies bound to fluorescently tagged antibodies, that are detected by a fluorescence activated cell sorter (FACS). Tumor endothelial cell marker antibodies, antibodies to EGFR, and optionally antibodies to endothelial cell markers, may be used in any appropriate combination to determine the EGFR status of the tumor endothelial cells.

Other Diagnostic Methods

An agent for detecting mutant EGFR protein is an antibody capable of binding to mutant EGFR, preferably an antibody with a detectable label. Antibodies can be polyclonal, or more preferably, monoclonal. An intact antibody, or a fragment thereof (e.g., F_{ab} or F_{(ab)2}) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect mutant EGFR mRNA, protein, or genomic DNA in a biological sample in vitro as well as in vivo. For example, in vitro techniques for detection of mutant EGFR mRNA include Northern hybridizations and in situ hybridizations. In vitro techniques for detection of mutant EGFR protein include enzyme linked immunosorbent assays (ELISAs), Western blots, immunoprecipitations, and immunofluorescence. In vitro techniques for detection of mutant EGFR genomic DNA include Southern hybridizations. Furthermore, in vivo techniques for detection of mutant EGFR protein include introducing into a subject a labeled anti-mutant EGFR protein antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject.

In another embodiment, the methods further involve obtaining a control biological sample from a control subject, contacting the control sample with a compound or agent capable of detecting mutant EGFR protein, mRNA, or genomic DNA, such that the presence of mutant EGFR protein, mRNA or genomic DNA is detected in the biological sample, and comparing the presence of mutant EGFR protein, mRNA or genomic DNA in the control sample with the presence of mutant EGFR protein, mRNA or genomic DNA in the test sample.

In another embodiment, the diagnostic assay is for mutant EGFR activity. In a specific embodiment, the mutant EGFR activity is a tyrosine kinase activity. One such diagnostic assay is for detecting EGFR-mediated phosphorylation of at least one EGFR substrate. Levels of EGFR activity can be assayed for, e.g., various mutant EGFR polypeptides, various tissues containing mutant EGFR, biopsies from cancer tissues suspected of having at least one mutant EGFR, and the like. Comparisons of the levels of EGFR activity in these various cells, tissues, or extracts of the same, can optionally be made. In one embodiment, high levels of EGFR activity in cancerous tissue is diagnostic for cancers that may be susceptible to treatments with one or more tyrosine kinase inhibitor. In related embodiments, EGFR activity levels can be determined between treated and untreated biopsy samples, cell lines, transgenic animals, or extracts from any of these, to determine the effect of a given treatment on mutant EGFR activity as compared to an untreated control.

ErbB2 protein exhibits different phosphorylation patterns depending on its heterodimerization partner. Antibodies specific for ErbB2 phosphorylation sites that are phosphorylated when the heterodimerization partner is EGFR may be used to detect the presence of ErbB2-EGFR heterodimers in tumor endothelial cells.

Method of Treating a Patient

In one embodiment, the invention provides a method for selecting a treatment for a patient affected by or at risk for developing cancer by determining the presence or absence of EGFR expression in tumor associated endothelial cells.

In certain embodiments, the presence of EGFR expression in tumor endothelial cells is indicative that an EGFR targeting treatment will be effective or otherwise beneficial (or more likely to be beneficial) in the individual. Stating that the treatment will be effective means that the probability of beneficial therapeutic effect is greater than in a person not having the appropriate presence of EGFR expression in tumor associated endothelial cells.

In one embodiment, the treatment involves the administration of a tyrosine kinase inhibitor. In particular, the tyrosine kinase inhibitor is an EGFR tyrosine kinase inhibitor. The treatment may involve a combination of treatments, including, but not limited to a tyrosine kinase inhibitor in combination with other tyrosine kinase inhibitors, chemotherapy, radiation, etc.

In one embodiment, detection of ErbB2 heterodimerization with EGFR in tumor endothelial cells indicates that an ErbB2 targeting treatment will be effective or otherwise beneficial or more likely to be beneficial in the individual.

Thus, in connection with the administration of a tyrosine kinase inhibitor, a drug which is “effective against” a cancer indicates that administration in a clinically appropriate manner results in a beneficial effect for at least a statistically significant fraction of patients, such as a improvement of symptoms, a cure, a reduction in disease load, reduction in tumor mass or cell numbers, extension of life, improvement in quality of life, or other effect generally recognized as positive by medical doctors familiar with treating the particular type of disease or condition.

Anti-EGFR Therapeutics

The present invention provides a method to direct treatment of a subject with a tumor, wherein the EGFR expression status of the endothelial cells associated with tumor is utilized for the direction of treatment, wherein positive EGFR expression status directs treatment of the subject towards administration of an EGFR targeting treatment. In one embodiment, a treatment is administered that targets both EGFR and ErbB2.

Inhibitors of EGFR include, but are not limited to, tyrosine kinase inhibitors such as quinazolines, such as PID 153035, 4-(3-chloroanilino)quinazoline, or CP-358,774, pyridopyrimidines, pyrimidopyrimidines, pyrrolopyrimidines, such as CGP 59326, CGP 60261 and CGP 62706, and pyrazolopyrimidines, 4-(phenylamino)-7H-pyrrolo[2,3-d]pyrimidines (Traxler et al., (1996) J. Med Chem 39:2285-2292), curcumin (diferuloyl methane) (Laxmin arayana, et al., (1995), Carcinogen 16:1741-1745), 4,5-bis (4-fluoroanilino) phthalimide (Buchdunger et al. (1995) Clin. Cancer Res. 1:813-821; Dinney et al. (1997) Clin. Cancer Res. 3:161-168); tyrphostins containing nitrothiophene moieties (Brunton et al. (1996) Anti Cancer Drug Design 11:265-295); the protein kinase inhibitor ZD-1 839 (AstraZeneca); CP-358774 (Pfizer, Inc.); PD-01 83805 (Warner-Lambert), EKB-569 (Torrance et al., Nature Medicine, Vol. 6, No. 9, September 2000, p. 1024), HKI-272 and HKI-357 (Wyeth); or as described in International patent application WO05/018677 (Wyeth); WO99/09016 (American Cyanamid); WO98/43960 (American Cyanamid); WO 98/14451; WO 98/02434; WO97/38983 (Warener Labert); WO99/06378 (Warner Lambert); WO99/06396 (Warner Lambert) ; WO96/30347 (Pfizer, Inc.); WO96/33978 (Zeneca); WO96/33977 (Zeneca); and WO96/33980 (Zeneca), WO 95/19970; U.S. Pat. App. Nos. 2005/0101618 assigned to Pfizer, 2005/0101617, 20050090500 assigned to OSI Pharmaceuticals, Inc.; all herein incorporated by reference. Further useful EGFR inhibitors are described in U.S. Pat. App. No. 20040127470, particularly in tables 10, 11, and 12, and are herein incorporated by reference.

EGFR-inhibiting agents include, but are not limited to, Gefitinib (compound ZD1839 developed by AstraZeneca UK Ltd.; available under the tradename IRESSA; hereinafter “IRESSA”) and Erlotinib (compound OSI-774 developed by Genentech, Inc. and OSI Pharmaceuticals, Inc.; available under the tradename TARCEVA; hereinafter “TARCEVA”); the monoclonal antibodies cetuximab (Erbitux; hnmClone Systems Inc/Merck KGaA), matuzumab (Merck KGaA) and anti-EGFR 22Mab (ImClone Systems Incorporated of New York, N.Y., USA), for egf/r3 MAb (Cuban Institute of Oncology; Hybridoma, 2001, Vol. 20, No. 2: 131-136), panitumumab/ABX-EGF (Abgenix/Cell Genesys), nimotuzumab ((TheraCIM-hR3) YM BioSciences Inc. Mississauga, Ontario, Canada), EMD-700, EMD-7200, EMD-5590 (Merck KgaA), E7.6.3 (Abgenix; Cancer Research 59, 1236-1243, 1999), Mab 806 (Ludwig Institute), MDX-103, MDX-447/H-477 (Medarex Inc. of Annandale, N.J., USA and Merck KgaA), and the compounds ZD-1834, ZD-1838 and ZD-1839 (AstraZeneca), PKI-166 (Novartis), PKI-166/CGP-75166 (Novartis), PTK 787 (Novartis), AEE788 (Novartis), CP 701 (Cephalon), leflunomide (Pharmacia/Sugen), CI-1033/PD-169414/PD-183805/Canertinib (Pfizer), CP-358774 (Pfizer), PD-168393, PD-158780, PD-160678 (Parke-Davis), CL-387,785 ((N-[4-[(3-bromophenyl)amino]-6-quinazolinyl]-2-butynamide; C. M. Discafani, et al.; Biochem. Pharmacol. 57:917 (1999)), BBR-1611 (Boehringer Mannheim GmbH/Roche), Naamidine A (Bristol Myers Squibb), RC-3940-II (Pharmacia), BIBX-1382 (Boehringer Ingelheim), OLX-103 (Merck & Co. of Whitehouse Station, N.J., USA), VRCTC-310 (Ventech Research), EGF fusion toxin (Seragen Inc. of Hopkinton, Mass.), DAB-389 (Seragen/Lilgand), ZM-252808 (Imperical Cancer Research Fund), RG-50864 (INSERM), LFM-A12 (Parker Hughes Cancer Center), WHI-P97 (Parker Hughes Cancer Center), GW-282974, GW2016 (Glaxo), KT-8391 (Kyowa Hakko) and EGFR Vaccine (York Medical/Centro de Immunologia Molecular (CIM)), EXEL 7647/EXEL 0999, XL647 (Exelixis), AG1478 (4-(3-Chloroanillino)-6,7-dimethoxyquinazoline), AG879 (3,5-Di-t-butyl-4-hydroxy-benzylidene)thiocyanoacetamide), ICR15, ICR16, and ICR80 (Int J Cancer. 1998 Jan. 19; 75(2):310-6.), ICR62 (Modjtahedi et al. Br J Cancer 1996; 73:228-35.), CGP 59326A (Novartis), BMS-599626 (Bristol-Myers Squibb)). These and other EGFR-inhibiting agents can be used in the present invention.

Some inhibitors of ErbB2 also inhibit EGFR and may be useful in the methods of the present invention. ErbB2 inhibitors include CI-1003, CP-724,714, CP-654577 (Pfizer, Inc.), GW-2016, GW-282974, and lapatinib/GW-572016 (Glaxo Wellcome plc), TAK-165 (Takeda), AEE788 (Novartis), EKB-569, HKI-272 and HKI-357 (Wyeth) (Wyeth-Ayerst), EXEL 7647/EXEL 0999 (EXELIXIS) and the monoclonal antibodies Trastuzumab (tradename HERCEPTIN), 2C4 (Genentech), AR-209 (Aronex Pharmaceuticals Inc. of The Woodlands, Tex., USA), pertuzumab (tradename OMNITARG; Genentech), BMS-599626 (Bristol-Myers Squibb) and 2B-1 (Chiron). For example those indicated in U.S. Pat. Nos. 6,867,201, 6,541,481, 6,284,764, 5,587,458 and 5,877,305; WO 98/02434, WO 99/35146, WO 99/35132, WO 98/02437, WO 97/13760, WO 95/19970, which are all hereby incorporated herein in their entireties by reference. The ErbB2 receptor inhibitor compounds and substance described in the aforementioned PCT applications, U.S. patents, and U.S. patent applications, as well as other compounds and substances that inhibit the ErbB2 receptor, can be used with the compound of the present invention in accordance with the present invention.

In another embodiment, compounds useful in the method of the present invention are antibodies which interfere with kinase signaling via EGFR, including monoclonal, chimeric, humanized, recombinant antibodies and fragment thereof which are characterized by their ability to inhibit the kinase activity of the EGFR and which have low toxicity.

Neutralizing antibodies are readily raised in animals such as rabbits or mice by immunization with an EGFR. Immunized mice are particularly useful for providing sources of B cells for the manufacture of hybridomas, which in turn are cultured to produce large quantities of anti-EGFR monoclonal antibodies. Chimeric antibodies are immunoglobin molecules characterized by two or more segments or portions derived from different animal species. Generally, the variable region of the chimeric antibody is derived from a non-human mammalian antibody, such as murine monoclonal antibody, and the immunoglobin constant region is derived from a human immunoglobin molecule. Preferably, both regions and the combination have low immunogenicity as routinely determined. Humanized antibodies are immunoglobin molecules created by genetic engineering techniques in which the murine constant regions are replaced with human counterparts while retaining the murine antigen binding regions. The resulting mouse-human chimeric antibody should have reduced immunogenicity and improved pharmacokinetics in humans. Examples of high affinity monoclonal antibodies and chimeric derivatives thereof, that are useful in the methods of the present invention, are described in the European Patent Application EP 186,833; PCT Patent Application WO 92/16553; and U.S. Pat. No. 6,090,923.

### Kits

In another embodiment of the present invention, kits useful for the detection of EGFR expression are disclosed. Such kits may include any or all of the following: assay reagents, buffers, specific nucleic acids or antibodies (e.g. full-size monoclonal or polyclonal antibodies, single chain antibodies (e.g., scFv), or other gene product binding molecules), and other hybridization probes and/or primers, and/or substrates for polypeptide gene products.

In addition, the kits may include instructional materials containing directions (i.e., protocols) for the practice of the methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### Screening for EGFR Targeting Agents

Test Compounds for Screening Targeting Agents

The term “agent” or “compound” as used herein and throughout the specification means any organic or inorganic molecule, including modified and unmodified nucleic acids such as antisense nucleic acids, RNAi, such as siRNA or shRNA, peptides, peptidomimetics, receptors, ligands, and antibodies.

In the methods of the present invention, a variety of test compounds and physical conditions from various sources can be screened for the ability of the compound to target EGFR and/or target ErbB2 in tumor endothelial cells that express EGFR. In one preferred embodiment, the ErbB2 targeting agent preferentially targets ErbB2 that is phosphorylated in a manner that is specific to ErbB2 when heterodimerized with EGFR. In another preferred embodiment, the targeting agent targets both EGFR and ErbB2.

Compounds to be screened can be naturally occurring or synthetic molecules. Compounds to be screened can also be obtained from natural sources, such as, marine microorganisms, algae, plants, and fungi. The test compounds can also be minerals or oligo agents. Alternatively, test compounds can be obtained from combinatorial libraries of agents, including peptides or small molecules, or from existing repertories of chemical compounds synthesized in industry, e.g., by the chemical, pharmaceutical, environmental, agricultural, marine, cosmetic, drug, and biotechnological industries. Test compounds can include, e.g., pharmaceuticals, therapeutics, agricultural or industrial agents, environmental pollutants, cosmetics, drugs, organic and inorganic compounds, lipids, glucocorticoids, antibiotics, peptides, proteins, sugars, carbohydrates; chimeric molecules, and combinations thereof.

Combinatorial libraries can be produced for many types of compounds that can be synthesized in a step-by-step fashion. Such compounds include polypeptides, proteins, nucleic acids, beta-turn mimetics, polysaccharides, phospholipids, hormones, prostaglandins, steroids, aromatic compounds, heterocyclic compounds, benzodiazepines, oligomeric N-substituted glycines and oligocarbamates. In the method of the present invention, the preferred test compound is a small molecule, nucleic acid and modified nucleic acids, peptide, peptidomimetic, protein, glycoprotein, carbohydrate, lipid, or glycolipid. In certain embodiments, the nucleic acid is DNA or RNA.

Large combinatorial libraries of compounds can be constructed by the encoded synthetic libraries (ESL) method described in Affymax, WO 95/12608, Affymax WO 93/06121, Columbia University, WO 94/08051, Pharmacopeia, WO 95/35503 and Scripps, WO 95/30642 (each of which is incorporated herein by reference in its entirety for all purposes). Peptide libraries can also be generated by phage display methods. See, e.g., Devlin, WO 91/18980. Compounds to be screened can also be obtained from governmental or private sources, including, e.g., the DIVERSet E library (16,320 compounds) from ChemBridge Corporation (San Diego, Calif.), the National Cancer Institute's (NCI) Natural Product Repository, Bethesda, Md., the NCI Open Synthetic Compound Collection, Bethesda, Md., NCI's Developmental Therapeutics Program, or the like.

Additionally, natural and synthetically produced libraries and compounds are readily modified through conventional chemical, physical, and biochemical means. In addition, known pharmacological agents may be subject to directed or random chemical modifications, such as acylation, alkylation, esterification, amidification, etc.

The compound formulations may conveniently be presented in unit dosage form, e.g., tablets and sustained release capsules, and in liposomes, and may be prepared by any methods well know in the art of pharmacy. (See, for example, Remington: The Science and Practice of Pharmacy by Alfonso R. Gennaro (Ed.) 20th edition, Dec. 15, 2000, Lippincott, Williams & Wilkins; ISBN: 0683306472.).

Screening Methods

Screening compounds for potential effectiveness in targeting EGFR or ErbB2 in tumor endothelial cells can be accomplished by a variety of means well known by a person skilled in the art.

To screen the compounds described above for ability to target EGFR, the test compounds should be administered to the test subject. In one preferred embodiment the test subject is a culture of tumor endothelial cells. The tumor endothelial cells may be a primary cell culture or an immortalized cell line. The tumor endothelial cells may be obtained from an animal, including but not limited to; a fish such as zebrafish, a rodent such as a mouse or a rat, a rabbit, a non-human primate and a human. In another embodiment, the test subject is an animal with tumor endothelial cells. The animal with tumor endothelial cells can be, but is not limited to, a fish such as a zebrafish, a rodent such as a mouse or a rat, a rabbit, a non-human primate, and a human.

The test compounds can be administered, for example, by diluting the compounds into the medium wherein the cell is maintained, mixing the test compounds with the food or liquid of the animal with tumor endothelial cells, topically administering the compound in a pharmaceutically acceptable carrier on the animal with the tumor endothelial cells, using three-dimensional substrates soaked with the test compound such as slow release beads and the like and embedding such substrates into the animal, or parenterally admininstering the compound. In some embodiments, the compounds are diluted into the media wherein the cell is maintained.

A variety of other reagents may also be included in the mixture. These include reagents such as salts, buffers, neutral proteins, e.g. albumin, detergents, etc. which may be used to facilitate optimal protein-protein and/or protein-nucleic acid binding and/or reduce non-specific or background interactions, etc. Also, reagents that otherwise improve the efficiency of the assay, such as protease inhibitors, nuclease inhibitors, antimicrobial agents, etc. may be used.

The language “pharmaceutically acceptable carrier” is intended to include substances capable of being coadministered with the compound and which allows the active ingredient to perform its intended function of preventing, ameliorating, arresting, or eliminating a disease(s) of the nervous system. Examples of such carriers include solvents, dispersion media, adjuvants, delay agents and the like. The use of such media and agents for pharmaceutically active substances is well known in the art. Any conventional media and agent compatible with the compound may be used within this invention.

The compounds can be formulated according to the selected route of administration. The addition of gelatin, flavoring agents, or coating material can be used for oral applications. For solutions or emulsions in general, carriers may include aqueous or alcoholic/aqueous solutions, emulsions or suspensions, including saline and buffered media. Parenteral vehicles can include sodium chloride, potassium chloride among others. In addition intravenous vehicles can-include fluid and nutrient replenishers, electrolyte replenishers among others.

Preservatives and other additives can also be present. For example, antimicrobial, antioxidant, chelating agents, and inert gases can be added (see, generally, Remington's Pharmaceutical Sciences, 16th Edition, Mack, 1980).

Screening for a compound that targets EGFR or ErbB2 can be accomplished using measurements of cell growth or cell death. Screening may be accomplished by using measurements of EGFR or ErbB2 transcription or translation. Screening may be accomplished by using measurements of EGFR or ErbB2 phosphorylation. The abovementioned screening approaches may be used individually or in combination.

To test targeting of EGFR or ErbB2 by the test compound, a biological sample may be obtained from the test subject. A “biological sample” refers to a cell or population of cells or a quantity of tissue or fluid from an animal. Most often, the sample has been removed, from an animal, but the term “biological sample” can also refer to cells or tissue analyzed in vivo, i.e. without removal from the animal. Often, a “biological sample” will contain cells from the animal, but the term can also refer to non-cellular biological material, such as non-cellular fractions of blood, saliva, or urine, that can be used to measure gene expression levels. Biological samples include, but are not limited to, tissue biopsies, scrapes (e.g. buccal scrapes), whole blood, plasma, serum, urine, saliva, cell culture, or cerebrospinal fluid. Preferred biological samples include tissue biopsies, cell culture. The sample can be obtained by removing a sample of cells from an animal, but can also be accomplished by using previously isolated cells (e.g. isolated by another person), or by performing the methods of the invention in vivo.

As noted above, screening assays are generally carried out in vitro, for example, in cultured cells, in a biological sample, or fractions thereof. For ease of description, cell cultures, biological samples, and fractions are referred to as “samples” below. The sample is generally derived from an animal (e.g., any of the research animals mentioned above), preferably a mammal, and more preferably from a human.

Screening assays to detect EGFR or ErbB2 transcription or expression are well known to the skilled artisan. Examples of such assays are described above in the section of the specification relating to diagnosis of EGFR expression.

Cell growth assays-are performed by methods well known in the art, e.g. those of Ferrara & Henzel, 1989, Nature 380:439-443, Gospodarowicz et al., 1989, Proc. Natl. Acad. Sci. USA 86:7311-7315, and or Claffey et al., 1995, Biochem. Biophys. Acta 1246:1-9.

Cell death assays may include those that qualify the promotion of apoptosis. In one embodiment, cell cultures administered the test compound maybe examined for the presence of apoptotic foci and compared to untreated control cell cultures. The extent to which apoptotic foci are found in the treated cell culture provides an indication of the therapeutic efficacy of the composition.

### Examples

### Example 1

Materials and Methods

Cell lines and reagents used. Isolation and characterization of melanoma, liposarcoma, skin, and adipose ECs and their culture conditions have been described previously (13). HUVECs and HMVECs were purchased from Cambrex Bio Science (Walkersville Md.) and grown in EGM2 media (Cambrex) at 5% CO2. SV40 T antigen immortalized MS 1 murine endothelial cell line (generous gift from Dr. Jack Arbiser, Emory University, Atlanta, Ga.) and MDA-MB-231 human breast carcinoma cells (purchased from American Type Culture Collection, Manassas, Va.) were cultured in DMEM (Gibco, Rockville, Md.) supplemented with 10% fetal bovine serum (FBS, Gibco) at 5% CO2. The melanoma cell line A375SM (generous gift from Dr. Isaiah Fidler, M.D. Anderson Cancer Center, Houston, Tex.) was cultured at 10% CO2 in MEM (Gibco) containing 10% FBS.

Isolation of breast carcinoma derived EC. A375SM melanoma xenografts in nude mice were obtained as described previously (13). MDA-MB-231 xenografts in female nude mice (8-10 weeks old, Charles River, Wilmington, Mass.) were obtained by injecting MDA-MB-231 tumor cells (2×106 cells/mouse) with 1:1 volume of matrigel (B.D. Biosciences, Bedford, Mass.) subcutaneously into the dorsal lateral flank. All of the animal procedures were performed in compliance with Children's Hospital Boston guidelines and approved by the Institutional Animal Care and Use Committee. When tumors reached approximately 1 cm in diameter, they were excised. Isolation of endothelial cells was performed as previously described using fluorescein isothiocyanate (FITC)-anti mouse CD31 antibody (Pharmingen, Boston Mass.) and magnetic cell sorting system (13). After subculture in the presence of diphtheria toxin to kill any remaining human tumor cells (15), the isolated cells were subjected to a second round of purification using FITC-BS1-B4 magnetic cell sorting (MACS, Miltenyi Biotec, Auburn, Calif.). The cells were cultured in EGM2-MV media (Cambrex) and purity was determined as described previously (13).

Immunoprecipitation (IP) and western blotting (WB). Prior to growth factor stimulations, cells were cultured in serum free media for 24 hr and incubated for 10 minutes at room temperature with EGF (100 ng/ml; R&D Systems, Minneapolis, Minn.) or NRG1-μl extra-cellular domain (50 ng/ml; R&D Systems). Cells were also treated with the EGFR kinase inhibitor, AG1478 (Calbiochem, San Diego, Calif.), prepared in DMSO, fifteen minutes prior to growth factor stimulation. Immunoprecipitation (IP) and western blotting (WB) were performed as previously described (16). The antibodies used for IP and WB of ErbB1-4 were: SC-03, SC-284, SC-285, and SC-283 respectively (Santa Cruz Biotechnology, Santa Cruz, Calif.). Receptor phosphorylation was detected using anti-phosphotyrosine antibody (mAb 4G10; Upstate, Lake Placid, N.Y.). Analysis of phosphorylated—EGFR on lysates was performed using phospho-1068 EGFR (Cell Signaling Technologies, Beverly Mass.). In addition antibodies to GAPDH (Chemicon, Temecula, Calif.), (β-Actin (Sigma-Aldrich, St Louis, Mo.), phospho-Erk1-2 (New England Biolab, Beverly Mass.), and Erk1 (Santa Cruz Biotechnology) were purchased.

Fluorescence-activated cell sorting (FACS). Indirect immunofluorescense to detect EGFR and ErbB3 expression was carried out in 1% paraformaldehyde fixed cells that were permeabilized with methanol. Cells were incubated at 40 C for 1 hr with anti-EGFR (SC-03) and anti-ErbB3 (SC-285) antibodies (Santa Cruz Biotechnology) followed by incubation with anti-rabbit Alexa 488 secondary antibody (Molecular Probe, Eugene, Oreg.) for 1 hr at 40 C. At least 10,000 cells per samples were analyzed on a FACS VantageSE flow cytometer using the Cell Quest software (Becton Dickinson, San Jose, Calif.).

Cell proliferation. Cells (4×103) were plated in triplicates into 96 well plates and allowed to adhere overnight. The cells were then switched to serum free media containing growth factors and/or AG1478 at indicated concentrations. Cells were cultured for 72 hr and cell proliferation was determined by addition of 0.42 mg/ml of 3-(4,5-dimethylthiazol-2-yl)-2,5-diphenyltetrazolium bromide (MTT) reagent (Sigma-Aldrich) for 4 hr prior to harvesting. Media was removed and cells were solubilized in DMSO and absorbance was measured at 570 nm. The averages of the triplicates were calculated and cell proliferation was determined as the percentage of absorbance of treated cells to the untreated cells. All the experiments were at least performed twice.

Reverse Transcription—PCR (RT-PCR). Total cellular RNA was isolated using the RNeasy miniprep kit with on-column DNAse treatment as per the manufacturer's protocols (Qiagen, Valencia, Calif.). Reverse transcription and amplification were performed as previously described using 31 amplification cycles (13). The primers used were ACCACAGTCCATGCCATCAC (SEQ ID NO:1) and TCCACCACCCTGTTGCTGTA (GAPDH; SEQ ID NO:2), AAAATGGTCCCCTCGGCT (SEQ ID NO:3) and TCTGGGCTCTTCAGACCA (TGFα ; SEQ ID NO:4), TGCGGGACCATGAAGCT (SEQ ID NO:5) and TCTCAGTGGGAATTAGTCA (HB-EGF; SEQ ID NO:6), TGTCCCCTGTCCCACGAT (SEQ ID NO:7) and AGCCTTGCTCTGTGCCCA (EGF; SEQ ID NO:8).

Immunohistochemistry. Cryosections (8-10 μm thick), embedded in OCT compound (Tissue-Tek, Torrance Calif.), were obtained from A375SM and MDA-MB-231 tumor xenografts and skin of nude mice (13), which were then fixed in acetone followed by acetone/chloroform (1:1 v/v). The sections were further treated with methanol for 5 minutes followed by incubation with 5% normal donkey serum (Jackson Immunoresearch, Westgrove, Pa.) for 30 minutes prior to staining. CD31 was detected using anti-mouse CD31 (Pharmingen) and anti-rat Phycoerythrin (PE) antibodies (Jackson Immunoresearch). EGF receptors were detected using anti-EGFR (Cell Signaling Technology), anti-phospho1068 EGFR (Cell Signaling Technology), and anti-ErbB3 (SC-285 Santa Cruz) with anti-rabbit FITC antibodies (Jackson Immunoresearch). Nuclei were stained with Hoechst 33258 (Sigma-Aldrich).

Results

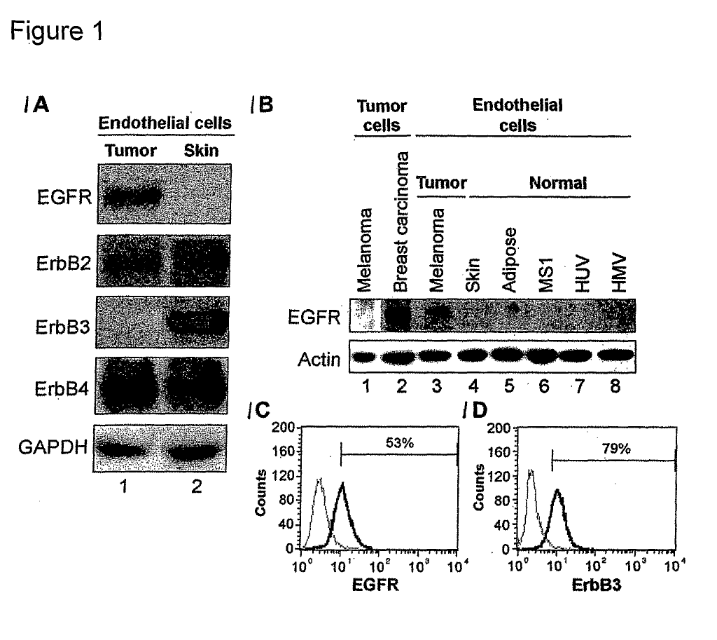

The EGF receptor family is differentially expressed in tumor versus normal endothelial cells. The distribution of the four EGF receptors in normal EC compared to tumor EC (purified from A375SM melanoma xenografts and devoid of human tumor cells) was investigated by western blotting (FIG. 1A). The tumor EC showed high expression of EGFR but undetectable levels of ErbB3, whereas their normal counterpart, skin EC, showed the opposite expression pattern (FIG. 1A). On the other hand, the other two EGF receptor family members, ErbB2 and ErbB4, did not show significant differences in expression between tumor and skin EC (FIG. 1A).

Expression of EGFR appeared to be specific for tumor EC since a number of EC lines derived from normal mouse and human tissue did not express EGFR (FIG. 1B, compare lanes 4-8 to lane 3). EGFR expression in the tumor cells themselves was variable. MDA-MB-231 breast tumor cells, expressed EGFR (FIG. 1B, lane 2) whereas, A375SM melanoma tumor cells did not express detectable EGFR (FIG. 1B, lane 1). Thus, in A375SM tumors, the tumor EC might be the major source of EGFR in the tumor. FACS analysis showed that 53% of tumor EC were positive for EGFR staining compared to IgG antibody control (FIG. 1C) whereas ErbB3 was expressed in 79% of skin EC (FIG. 1D).

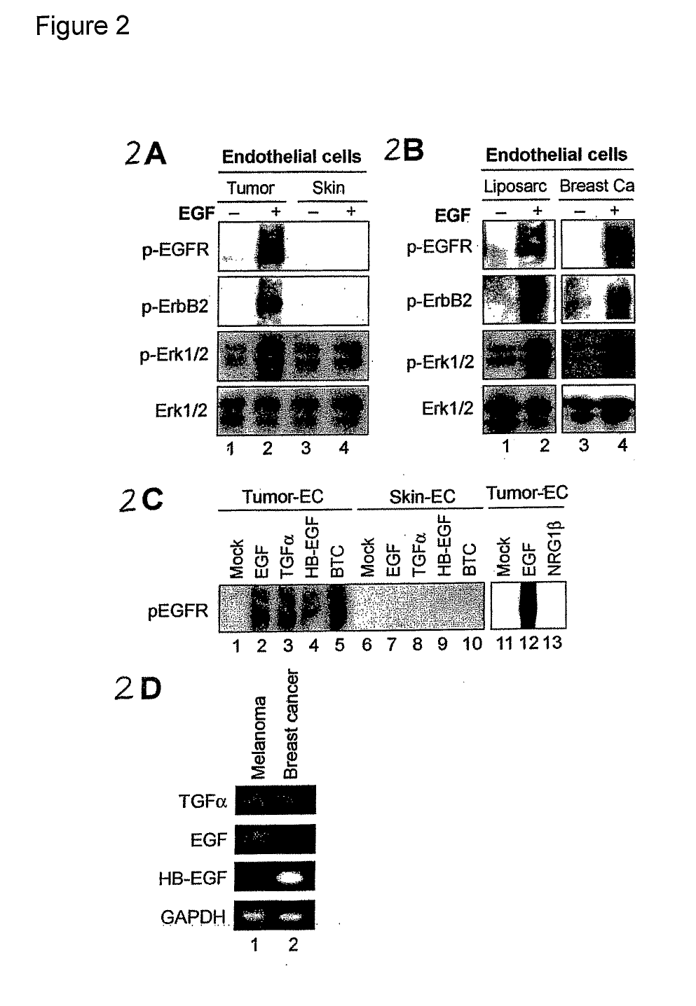

EGF activates EGFR and ErbB2 in tumor but not normal EC. The EGFR expression profile suggests that tumor, but not normal, EC would be activated in response to EGF or any EGFR ligand. EGF stimulation of tumor EC after serum starvation induced a robust increase in EGFR phosphorylation (FIG. 2A compare lane 1 to 2). As expected, skin EC, which did not express EGFR did not show any inducible EGFR phosphorylation (FIG. 2A, compare lane 3 to 4). EGF receptors form heterodimers, with ErbB2 being the preferred dimerization partner (17). ErbB2, which does not bind directly to any known ligands, can be activated by heterodimerizing with ligand bound EGFR. As might be expected from these considerations, addition of EGF increased ErbB2 phosphorylation (p-ErbB2) in tumor EC (FIG. 2A lane 2) but not skin EC (FIG. 2A lane 4). Activation of the EGFR:ErbB2 complex in the presence of EGF was accompanied by an increase in phospho-Erk1/2 levels indicating that receptor activation was able to couple to the MAPK signaling cascade (FIG. 2A).

Besides A375SM melanoma EC, EGF induced EGFR tyrosine phosphorylation and subsequent MAPK activation in liposarcoma EC and in MDA-MB-231 breast carcinoma EC (FIG. 2B, compare lane 1 to 2 and lane 3 to 4). ErbB2 was also activated in response to EGF in breast carcinoma and liposarcoma EC (FIG. 2B, compare lane 1 to 2 and lane 3 to 4).

EGF was not the only activator of EGFR in tumor EC. The EGFR ligands TGFα, HB-EGF, and BTC also promoted EGFR activation in tumor EC (FIG. 2C, compare lanes 2-5 to lane 1). As expected, none of these EGF family ligands activated EGFR in normal skin EC (FIG. 2C, compare lanes 7-10 to 6). In contrast, NRG1β, a ligand for ErbB3 and ErbB4, but not EGFR, was not able to activate EGFR in tumor EC (FIG. 2C compare lanes 12 and 13 to lane 11). EGFR ligands, including TGFα, EGF, and HB-EGF were expressed in A375SM and MDA-MB-231 tumor cells (FIG. 2D), suggesting that paracrine interactions between the tumor cells and the tumor vasculature could occur.

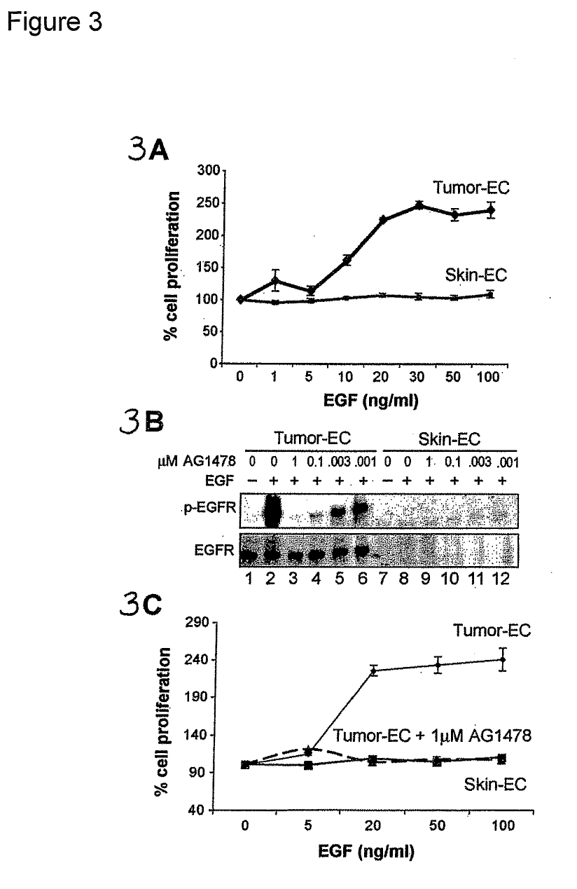

EGF stimulates tumor EC proliferation, which is inhibited by an EGFR kinase inhibitor. EGF induced a dose-dependent increase in cell proliferation in tumor EC (FIG. 3A). A 2.5 fold increase in cell proliferation was observed at a dose of 20 ng/ml of EGF. Hence, EGFR activation is sufficient for stimulating proliferation of tumor EC. In contrast, skin EC did not respond to EGF (FIG. 3A).

AG1478 is a small molecule kinase inhibitor with a high specificity for EGFR (18). AG1478 inhibited EGF-induced phosphorylation of EGFR in tumor EC in a dose-dependent manner (FIG. 3B, compare lanes 2-6 to lane 1) but had no effect on skin EC (FIG. 3B, lanes 7-12). AG1478, at 1 μM, was sufficient to completely inhibit the dose-dependent increase in tumor EC cell proliferation in response to EGF (FIG. 3C). IRESSA® (Gefitinib, ZD1839), another EGFR kinase inhibitor, was also able to inhibit EGFR tyrosine phosphorylation and inhibit EGF-induced tumor EC proliferation.

Expression of EGFR but not ErbB3 in tumor EC in vivo. Immunohistochemistry (IHC) of EGFR expression in tumor tissue sections was performed to eliminate the possibility that the EGFR expression in tumor EC was an artifact of cell culture conditions. Tumors were obtained from subcutaneous injections of A375SM melanoma cells and MDA-MB-231 breast carcinoma cells in athymic mice (data not shown). These parental tumors were the same ones used to isolate the tumor EC described in FIG. 1A. Mouse skin sections were also analyzed (data not shown). Frozen sections (8-10 μm) were double immuno-labeled for CD31, a marker for EC (red fluorescence), and for EGFR/p-EGFR/ErbB3 (green fluorescence). Co-localization was observed in the merged images in yellow.

Fluorescent microscopy showed that melanoma EGFR and CD31 positive blood vessels co-localized (data not shown). Furthermore, the CD31 positive blood vessels co-localized with phosphorylated EGFR (data not shown) indicating that the tumor EC express activated EGFR in vivo. Rabbit IgG controls showed no co-localization with CD31 staining. Interestingly, the melanoma tumor cells, as opposed to the melanoma EC did not stain with the EGFR antibody suggesting that, in these tumors, EGFR expression was mostly restricted to the endothelial compartment. These results are consistent with the western blot analysis shown in FIG. 1B. Co-localization of EGFR staining with CD31 positive EC was also observed in xenografts of MDA-MB-231 breast carcinomas (data not shown). Unlike the melanoma, both breast carcinoma tumor cells and EC were EGFR positive. In contrast, very little co-localization of EGFR and CD31 was evident in sections of mouse skin (data not shown). The hair follicle cells in the mouse skin serve as a positive control for EGFR staining (19).

Western blot analysis had shown that A375SM melanoma EC did not express detectable ErbB3 compared to skin EC (FIG. 1A). This was confirmed by IHC analysis of A375SM melanoma tissues, which showed a lack of ErbB3 co-localization with CD31 positive blood vessels (data not shown). In contrast, CD31 and ErbB3 signal co-localized in mouse skin tissue sections (data not shown).

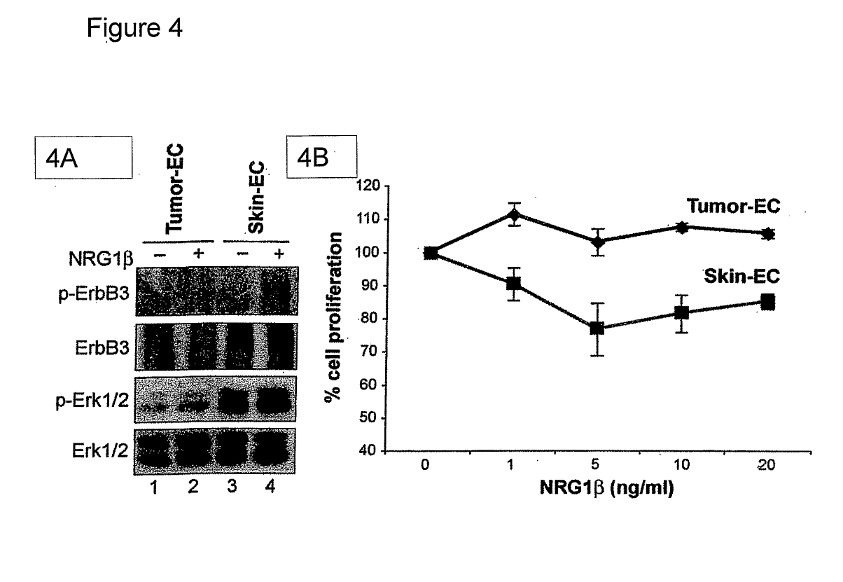

NRG activates ErbB3 signaling in normal EC and inhibits their growth. In FIG. 1A, it was shown that normal skin EC expressed ErbB3 but not EGFR. As expected, NRG1β did not activate ErbB3 in tumor EC (FIG. 2C, lane 13, FIG. 4A, lane 2). On the other hand, NRG1β did stimulate increased ErbB3 phosphorylation in skin EC (FIG. 4A, lane 4). NRG activation in several cell types is associated with growth inhibition and differentiation (20). NRG1β induced dose-dependent inhibition of cell growth in skin EC but not in tumor EC (FIG. 4B). A maximum of 25% growth inhibition was observed at a dose of 5 ng/ml (0.2 nM) NRG1β.

Discussion

Evidence is presented that tumor EC are markedly different from normal EC in expression of EGF receptors and in their response to EGF family members and to EGFR kinase inhibitors. Comparative analysis of the expression profiles of the four EGF receptors shows that tumor EC express EGFR, ErbB2, and ErbB4, whereas, normal EC express ErbB2, ErbB3 and ErbB4. Thus, there is a switch in which tumor EC express EGFR rather than ErbB3, opposite to the pattern of expression which occurs in normal EC. EGFR expression was evident in several tumor-derived EC lines tested including melanoma, breast carcinoma and liposarcoma EC, but not in several normal EC lines tested including skin, adipose, HUV, HMV and MS1 EC. Several previous studies have reported an absence of EGFR expression in HUVEC consistent with our observation (14, 21). However, EGFR expression in HUVEC has been recently reported (22). Analysis of tumor xenograft sections confirmed the western blot profiles. EGFR was co-localized by immunohistochemistry with the EC marker, CD31, in melanoma and breast carcinoma tumor sections. Furthermore, EGFR was activated in vivo in melanoma xenografts as detected by anti-phospho-EGFR antibodies. On the other hand, ErbB3 did not co-localize with CD31 in the tumor sections but did in skin sections.

The expression of EGFR in tumor, but not normal, EC has a number of consequences. One is that the tumor EC are targets for EGF, TGFα, BTC, and HB-EGF but not for NRG. EGF induces tumor EC EGFR tyrosine phosphorylation, activates downstream MAPK signaling pathway, and stimulates their proliferation. EGF binding to EGFR also activates its heterodimerizing partner ErbB2, an oncogene. Thus, in tumor EC, two EGF receptor types are activated by EGF. In contrast, EGFR ligands are unable to elicit any of these responses in normal skin EC, as expected, since normal EC do not express EGFR.

Interestingly, skin EC, but not tumor EC express ErbB3, suggesting that EC switch from being NRG responsive to EGF responsive as they encounter the tumor microenvironment. NRG, the ligand for ErbB3, activates the receptor in normal EC but not tumor EC. In the case of skin EC, NRG inhibits proliferation. There is no such inhibition in tumor EC since they do not express ErbB3. Thus, tumor EC may promote angiogenesis in two ways, by enhancing EGFR expression thereby increasing their ability to proliferate, and by losing ErbB3 expression, which would be growth inhibitory. In support, it has been shown that in a non-transformed breast epithelial cell line MCF10A, NRG signaling mediated via ErbB2 and ErbB3 was associated with a strong anti-proliferative response (23). Furthermore, a tumorigenic variant of MCF10A, MCF10CA, has reduced levels of ErbB3 and responds to NRG by cellular proliferation.

Another important consequence is that tumor EC, but not normal EC, are targets for anti-EGF receptor drugs, for example, EGFR kinase inhibitors. Tumors produce EGF family ligands such as TGFα and HB-EGF, and these growth factors have been suggested to stimulate autocrine and paracrine proliferation of tumor cells expressing EGFR (24). EGFR kinase inhibitors block tumor cell proliferation and tumor growth (6). We have found that EGFR kinase inhibitors affect EGF-tumor EC interactions as well. They completely inhibit EGF-induced tumor EC proliferation but have no effect on skin EC, which do not express EGFR. Gefitinib (IRESSA®), which is marketed for treating NSCLC patients, was also able to inhibit EGF-induced cell proliferation of tumor EC. These results are significant since they suggest that, in vivo, EGFR kinase inhibitors will target the tumor vasculature but not the normal vasculature, a specificity important for anti-angiogenesis drug design. These results also suggest that tumor EC might be a more appropriate pre-clinical model than HUVEC for studying anti-angiogenesis therapies such as EGF receptor kinase inhibitors. This may be one of the first demonstrations that anti-EGFR therapeutics directly target tumor derived EC. A375SM melanoma cells are of interest since they express very little tumor cell-associated EGFR but abundant EC-associated EGFR. In this tumor, the lack of EGFR on tumor cells makes the identification of EGFR on EC relatively unambiguous.

Another consequence of EGFR expression in tumor EC is that ErbB2/HER2 is also activated in response to EGF stimulation. Since, ErbB2 activation requires dimerization with a ligand binding receptor, and since EGF binds only to EGFR, a heterodimer of EGFR:ErbB2 must be activated in tumor EC. Signaling through the EGFR:ErbB2 heterodimer is more potent due to delayed endocytosis of the activated receptor (25). Transformation of tumor cells requires both EGFR as well as ErbB2 expression (26). In addition, co-expression of EGFR with ErbB2 in breast tumors is associated with a worse patient prognosis than single receptor expression (27). EGFR-positive patients with high levels of ErbB2 respond better to EGFR kinase inhibitors (28). Hence, the activation of EGFR:ErbB2 heterodimer signaling in tumor EC may provide more effective targets for EGFR kinase inhibitors as well as suggesting that anti-ErbB2 therapeutics (e.g. Herceptin) may also be efficacious in inhibiting tumor angiogenesis.

A novel finding is that normal EC express ErbB3, but tumor EC do not. As expected, NRG stimulates phosphorylation of ErbB3 in skin EC but not tumor EC. NRG has several biological functions including stimulating proliferation, differentiation, growth arrest, apoptosis, and endothelial to mesenchymal transitions (29, 30). In normal EC, we find that activation by NRG1β at 1-20 ng/ml results in their growth inhibition. In support, it has been reported that addition of NRG2 to HUVEC and HMVEC results in growth inhibition in a dose range of 1-10 ng/ml (31). Both studies use the NRG form containing EGF and the immunoglobulin domain. The immunoglobulin domain is critical for growth inhibition (31). Another study showed cell proliferation of HUVEC in response to NRG1β (14). However, these results could be due to their use of the NRG1β form lacking the immunoglobulin domain. NRG is a ligand for both ErbB3 and ErbB4. It is plausible that the growth inhibition observed in response to NRG in normal EC is acting not via ErbB3 but via ErbB4, which is also expressed in these cells. However, tumor EC, which express ErbB4 but do not express ErbB3, are not growth inhibited by NRG activation. These results suggest that the NRG-induced growth inhibition acts via the ErbB3 receptor.

EGFR expression in EC has been reported previously by immunohistochemical analysis of tumor xenografts of pancreatic and renal tumors (9, 12, 22). In these studies, EGFR kinase inhibitor treatment of mice bearing these tumors showed decreased p-EGFR expression and a concomitant increase of apoptosis in tumor associated EC as determined by immunohistochemical analysis. However, whether these EGFR kinase inhibitors block angiogenesis directly via EC-EGFR or indirectly by suppressing VEGF (10) is not clear. Our results suggest that EGFR kinase inhibitors have a direct inhibitory effect on tumor EC proliferation. Using a molecular approach, we have shown for the first time enhanced ErbB2 and MAPK activity in tumor EC in response to EGF and resistance to inhibitory activity of NRG due to loss of ErbB3.

Our tumor EC EGFR studies has clinical significance. One, is that anti-EGFR therapeutics target the tumor vasculature specifically based on our findings that tumor EC but not normal EC express EGFR. Patients eligible to receive anti EGFR drugs are screened by positive immunoreactivity to EGFR (32). However, a study in colon cancer patients showed that patients who had scores of 0 out of a 3+ scoring system for EGFR immunoreactivity in their tumors responded to Cetuximab (a monoclonal antibody against EGFR) (33). Our work indicates that determining EGFR immunoreactivity in tumor endothelium in addition to the tumor cells will help identify patients that have previously been determined as being ineligible for EGFR therapeutics.

### Example 2

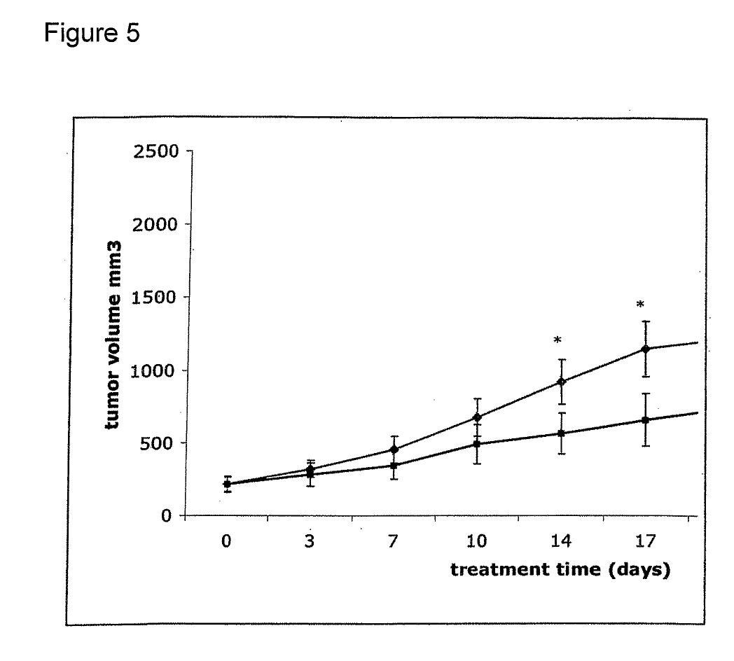

In the A375SM melanoma xenograft model in nude mice, the tumor cells are EGFR-negative, whereas the EC derived from these tumors are EGFR-positive. We used this model to determine whether anti-EGFR drugs (e.g. IRESSA®/Gefitinib, Astrazeneca) may be effective in inhibiting tumors where the tumor cells are negative for the receptor and thus directly target the EC. A375SM melanoma tumor cells were injected subcutaneously into nude mice. When the tumors reached an average volume of 200 mm³ the mice were randomized into a control and Iressa treatment groups. The mice were treated daily p.o. with gefitinib (150 mg/kg) or with the vehicle. Tumor volumes were measured every 4 days. Shown in FIG. 5 is the tumor volume from the start of treatment (day 0) until the end of treatment (day 28) for the Gefitinib (black, n=11) and Control groups (gray, n=11). Gefitinib treatment results in a 43% inhibition of growth in tumor volume.

### Example 3

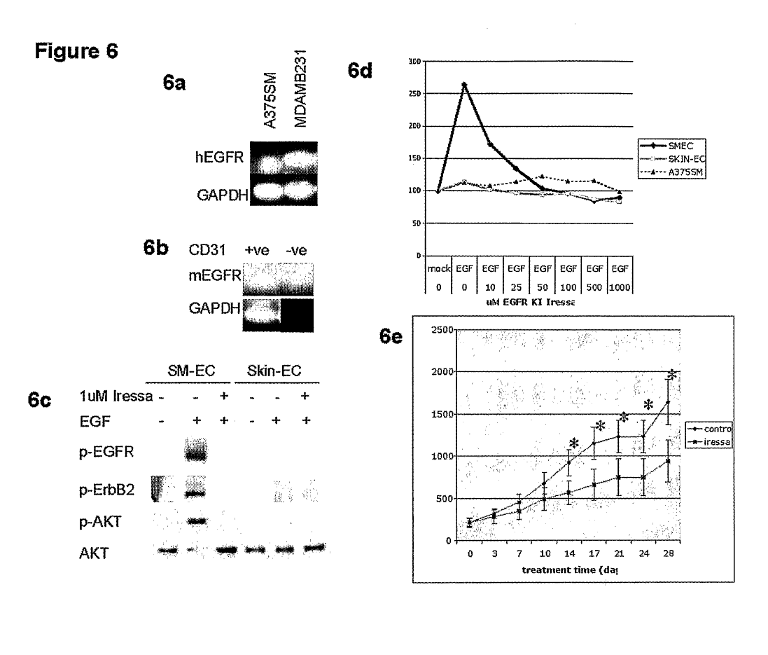

As previously shown herein, that whereas the A375SM tumor cells themselves do not express EGFR in vitro; EC derived from these tumors express EGFR and activation of this receptor results in EC cell proliferation. Also, EGFR staining of melanoma tumor sections showed that EGFR was mainly localized to the tumor vessels. To ensure that the main source of EGFR in tumors is EC we performed RT-PCR using human specific primers for EGFR on mRNA obtained from the tumor xenograft. Melanoma xenografts do not show expression of human EGFR transcripts (FIG. 6a). MDA-MB231 tumor cells provide a positive control for human EGFR amplification. Mouse specific primers easily detected EGFR transcripts, suggesting that the main source of EGFR is from the host stroma (FIG. 6b). In addition, mouse EGFR transcripts are mainly expressed in the CD31 positive fraction of cells isolated from these tumors (FIG. 6b). The CD31 positive fraction was obtained using EC isolation protocol previously described using MACS bead that bind to CD31 labeled cells. The negative fraction is derived from the flow through from the column that does not bind the CD31 labeled cells. We have previously shown that AG1478 inhibits EGFR activation on melanoma EC and completely inhibits EGF dependent proliferation observed in these cells. Similarly, we show that Iressa (1 uM) inhibits EGF induced phosphorylation of EGFR (FIG. 6c). In addition, Iressa inhibits EGF induced ErbB2 and AKT phosphorylation in these cells. Iressa inhibits EGF-dependent cell proliferation of melanoma EC in a dose dependent manner but not of normal skin EC or A375SM tumor cells (FIG. 6d). Daily administration of Iressa to mice bearing A375SM xenografts resulted in a 48% tumor growth inhibition after 4 weeks of treatment (FIG. 6e). Statistically significant differences based on students t-test analysis of p-value less than 0.05 was observed from day 14 of treatₘₑₙt onwards.

The references cited below and throughout the specification are incorporated herein in their entirety by reference.

### REFERENCES

1. Riese, D. J., 2nd and Stern, D. F. Specificity within the EGF family/ErbB receptor family signaling network. Bioessays, 1998, 20: 41-48.

2. Yarden, Y. and Sliwkowski, M. X. Untangling the ErbB signaling network. Nat Rev Mol Cell Biol, 2001, 2: 127-137.

3. Olayioye, M. A., Neve, R. M., Lane, H. A., and Hynes, N. E. The ErbB signaling network: receptor heterodimerization in development and cancer. Embo J, 2000, 19: 3159-3167.

4. Stern, D. F. Tyrosine kinase signalling in breast cancer: ErbB family receptor tyrosine kinases. Breast Cancer Res, 2000, 2: 176-183.

5. Ivanov, V. N. and Hei, T. K. Combined treatment with EGFR inhibitors and arsenite upregulated apoptosis in human EGFR-positive melanomas: a role of suppression of the PI3K-AKT pathway. Oncogene, 2005, 24: 616-626.

6. Hynes, N. E. and Lane, H. A. ERBB receptors and cancer: the complexity of targeted inhibitors. Nat Rev Cancer, 2005, 5: 341-354.

7. Baselga, J. and Arteaga, C. L. Critical update and emerging trends in epidermal growth factor receptor targeting in cancer. J Clin Oncol, 2005, 23: 2445-2459.

8. Izumi, Y., Xu, L., di Tomaso, E., Fukumura, D., and Jain, R. K. Tumour biology: herceptin acts as an anti-angiogenic cocktail. Nature, 2002, 416: 279-280.

9. Kedar, D., Baker, C. H., Killion, J. J., Dinney, C. P., and Fidler, I. J. Blockade of the epidermal growth factor receptor signaling inhibits angiogenesis leading to regression of human renal cell carcinoma growing orthotopically in nude mice. Clin Cancer Res, 2002, 8: 3592-3600.

10. Kumar, R. and Yarmand-Bagheri, R. The role of HER2 in angiogenesis. Semin Oncol, 2001, 28: 27-32.

11. Ferrara, N., Gerber, H. P., and LeCouter, J. The biology of VEGF and its receptors. Nat Med, 2003, 9: 669-676.

12. Baker, C. H., Kedar, D., McCarty, M. F., Tsan, R., Weber, K. L., Bucana, C. D., and Fidler, I. J. Blockade of epidermal growth factor receptor signaling on tumor cells and tumor-associated endothelial cells for therapy of human carcinomas. Am J Pathol, 2002, 161: 929-938.

13. Hida, K., Hida, Y., Amin, D. N., Flint, A. F., Panigrahy, D., Morton, C. C., and Klagsbrun, M. Tumor-associated endothelial cells with cytogenetic abnormalities. Cancer Res, 2004, 64: 8249-8255.

14. Russell, K. S., Stern, D. F., Polverini, P. J., and Bender, J. R. Neuregulin activation of ErbB receptors in vascular endothelium leads to angiogenesis. Am J Physiol, 1999, 277: H2205-2211.

15. Arbiser, J. L., Larsson, H., Claesson-Welsh, L., Bai, X., LaMontagne, K., Weiss, S. W., Soker, S., Flynn, E., and Brown, L. F. Overexpression of VEGF 121 in immortalized endothelial cells causes conversion to slowly growing angiosarcoma and high level expression of the VEGF receptors VEGFR-1 and VEGFR-2 in vivo. Am J Pathol, 2000, 156: 1469-1476.

16. Amin, D. N., Tuck, D., and Stern, D. F. Neuregulin-regulated gene expression in mammary carcinoma cells. Exp Cell Res, 2005, 309: 12-23.

17. Graus-Porta, D., Beerli, R. R., Daly, J. M., and Hynes, N. E. ErbB-2, the preferred heterodimerization partner of all ErbB receptors, is a mediator of lateral signaling. Embo J, 1997, 16: 1647-1655.

18. Yaish, P., Gazit, A., Gilon, C., and Levitzki, A. Blocking of EGF-dependent cell proliferation by EGF receptor kinase inhibitors. Science, 1988, 242: 933-935

19. Nanney, L. B., Stoscheck, C. M., King, L. E., Jr., Underwood, R. A., and Holbrook, K. A. Immunolocalization of epidermal growth factor receptors in normal developing human skin. J Invest Dermatol, 1990, 94: 742-748.

20. Burden, S. and Yarden, Y. Neuregulins and their receptors: a versatile signaling module in organogenesis and oncogenesis. Neuron, 1997, 18: 847-855.

21. Kim, H. S., Shin, H. S., Kwak, H. J., Cho, C. H., Lee, C. O., and Koh, G. Y. Betacellulin induces angiogenesis through activation of mitogen-activated protein kinase and phosphatidylinositol 3′-kinase in endothelial cell. Faseb J, 2003, 17: 318-320.

22. Sini, P., Wyder, L., Schnell, C., O'Reilly, T., Littlewood, A., Brandt, R., Hynes, N. E., and Wood, J. The antitumor and antiangiogenic activity of vascular endothelial growth factor receptor inhibition is potentiated by ErbB 1 blockade. Clin Cancer Res, 2005, 11: 4521-4532.

23. Li, Q., Ahmed, S., and Loeb, J. A. Development of an autocrine neuregulin signaling loop with malignant transformation of human breast epithelial cells. Cancer Res, 2004, 64: 7078-7085.

24. Salomon, D. S., Brandt, R., Ciardiello, F., and Normanno, N. Epidermal growth factor-related peptides and their receptors in human malignancies. Crit Rev Oncol Hematol, 1995, 19: 183-232.

25. Huang, G., Chantry, A., and Epstein, R. J. Overexpression of ErbB2 impairs ligand-dependent downregulation of epidermal growth factor receptors via a post-transcriptional mechanism. J Cell Biochem, 1999, 74: 23-30.

26. Kokai, Y., Meyers, J. N., Wada, T., Brown, V. I., LeVea, C. M., Davis, J. G., Dobashi, K., and Greene, M. I. Synergistic interaction of p185 c-neu and the EGF receptor leads to transformation of rodent fibroblasts. Cell, 1989, 58: 287-292.

27. Suo, Z., Risberg, B., Kalsson, M. G., Willman, K., Tierens, A., Skovlund, E., and Nesland, J. M. EGFR family expression in breast carcinomas. c-erbB-2 and c-erbB-4 receptors have different effects on survival. J Pathol, 2002, 196: 17-25.

28. Cappuzzo, F., Varella-Garcia, M., Shigematsu, H., Domenichini, I., Bartolini, S., Ceresoli, G. L., Rossi, E., Ludovini, V., Gregorc, V., Toschi, L., Franklin, W. A., Crino, L., Gazdar, A. F., Bunn, P. A., Jr., and Hirsch, F. R. Increased HER2 gene copy number is associated with response to gefitinib therapy in epidermal growth factor receptor-positive non-small-cell lung cancer patients. J Clin Oncol, 2005, 23: 5007-5018.

29. Falls, D. L. Neuregulins: functions, forms, and signaling strategies. Exp Cell Res, 2003, 284: 14-30.

30. Camenisch, T. D., Schroeder, J. A., Bradley, J., Klewer, S. E., and McDonald, J. A. Heart-valve mesenchyme formation is dependent on hyaluronan-augmented activation of ErbB2-ErbB3 receptors. Nat Med, 2002, 8: 850-855.

31. Nakano, N., Higashiyama, S., Ohmoto, H., Ishiguro, H., Taniguchi, N., and Wada, Y. The N-terminal region of NTAK/neuregulin-2 isoforms has an inhibitory activity on angiogenesis. J Biol Chem, 2004, 279: 11465-11470.

32. Dei Tos, A. P. and Ellis, I. Assessing epidermal growth factor receptor expression in tumours: what is the value of current test methods? Eur J Cancer, 2005, 41: 1383-1392.

33. Chung, K. Y., Shia, J., Kemeny, N. E., Shah, M., Schwartz, G. K., Tse, A., Hamilton, A., Pan, D., Schrag, D., Schwartz, L., Klimstra, D. S., Fridman, D., Kelsen, D. P., and Saltz, L. B. Cetuximab shows activity in colorectal cancer patients with tumors that do not express the epidermal growth factor receptor by immunohistochemistry. J Clin Oncol, 2005, 23: 1803-1810.

We claim:

1. A method for determining the likelihood of effectiveness of an EGFR targeting treatment in a subject affected with a tumor comprising: detecting the presence or absence of EGFR expression in endothelial cells associated with the tumor, wherein the presence of EGFR expression indicates that the EGFR targeting treatment is likely to be effective.

2. The method of claim 1, wherein the tumor does not express EGFR.

3. The method of claim 1, wherein EGFR expression is evaluated in a biological sample from said subject.

4. The method of claim 3, wherein the biological sample comprises a tumor.

5. The method of claim 3, wherein the biological sample comprises tumor endothelial cells.

6. The method of claim 1, wherein said tumor is selected from the group consisting of gastrointestinal cancer, prostate cancer, ovarian cancer, breast cancer, head and neck cancer, lung cancer, non-small cell lung cancer, cancer of the nervous system, kidney cancer, retinal cancer, skin cancer, stomach cancer, liver cancer, pancreatic cancer, genital-urinary cancer, prostate cancer, colorectal cancer and bladder cancer.

7. The method of claim 1, wherein EGFR expression is determined by RT-PCR, quantitative RT-PCR, Northern Blot, microarray based expression analysis, fluorescence in situ hybridization (FISH), ligase chain reaction (LCR), transcription amplification, self-sustained sequence replication, ELISA, western blot, antibody staining, immunohistochemistry, and/or immunofluorescence.

8. The method of claim 1, wherein EGFR expression is determined by immunohistochemistry.

9. A method of treating a subject affected with or at risk for developing cancer, comprising detecting the presence or absence of EGFR expression in endothelial cells associated with a tumor in said subject, wherein the subject is administered an EGFR targeting treatment if the presence of the said EGFR expression is detected.

10. The method of claim 9, wherein the tumor does not express EGFR.

11. The method of claim 9, wherein the EGFR targeting treatment is a tyrosine kinase inhibitor.

12. The method of claim 9, wherein the EGFR targeting treatment is an EGFR inhibitor.

13. The method of claim 12, wherein the EFGR inhibitor is selected from the group consisting of a small molecule inhibitor, a competitive inhibitor, a nucleic acid, an antibody, an antibody fragment, or an aptamer.

14. The method of claim 12, wherein the EGFR inhibitor is gefitinib, erlotinib, cetuximab, matuzumab, panitumumab, AEE788; CI-1033, HKI-272, HKI-357 or EKB-569.

15. A method for determining the likelihood of effectiveness of an EGFR targeting treatment in a subject affected with a tumor comprising: detecting the presence or absence of EGFR expression in endothelial cells associated with the tumor, wherein the presence of EGFR expression indicates that the ErbB2 targeting treatment is likely to be effective.

16. The method of claim 15, wherein the tumor does not express EGFR.

17. The method of claim 15, wherein EGFR expression is evaluated in a biological sample from said subject.

18. The method of claim 17, wherein the biological sample comprises a tumor.

19. The method of claim 17, wherein the biological sample comprises tumor endothelial cells.

20. The method of claim 15, wherein said tumor is selected from the group consisting of gastrointestinal cancer, prostate cancer, ovarian cancer, breast cancer, head and neck cancer, lung cancer, non-small cell lung cancer, cancer of the nervous system, kidney cancer, retinal cancer, skin cancer, stomach cancer, liver cancer, pancreatic cancer, genital-urinary cancer, prostate cancer, colorectal cancer and bladder cancer.

21. The method of claim 15, wherein EGFR expression is determined by. RT-PCR, quantitative RT-PCR, Northern Blot, microarray based expression analysis, fluorescence in situ hybridization (FISH), ligase chain reaction (LCR), transcription amplification, self-sustained sequence replication, ELISA, western blot, antibody staining, immunohistochemistry, and/or immunofluorescence.

22. The method of claim 15, wherein EGFR expression is determined by immunohistochemistry.

23. A method of treating a subject affected with or at risk for developing cancer, comprising detecting the presence or absence of EGFR expression in endothelial cells associated with a tumor in said subject, wherein the subject is administered an ErbB2 targeting treatment if the presence of the said EGFR expression is detected.

24. The method of claim 23, wherein the tumor does not express EGFR.

25. The method of claim 23, wherein the ErbB2 targeting treatment is a tyrosine kinase inhibitor.

26. The method of claim 23, wherein the ErbB2 targeting treatment is an ErbB2 inhibitor.

27. The method of claim 26, wherein the ErbB2 inhibitor is selected from the group consisting of a small molecule inhibitor, a competitive inhibitor, a nucleic acid, an antibody, an antibody fragment, or an aptamer.

28. The method of claim 26, wherein the ErbB2 inhibitor is trastuzumab, pertuzumab, lapatinib, HKI-272 or HKI-357.

29. A method to direct treatment of a subject with a tumor, wherein the EGFR expression status of the endothelial cells associated with tumor is utilized for the direction of treatment, wherein positive EGFR expression status directs treatment of the subject towards administration of an EGFR targeting treatment.

30. A method to direct treatment of a subject with a tumor, wherein the EGFR expression status of the endothelial cells associated with tumor is utilized for the direction of treatment, wherein positive EGFR expression status directs treatment of the subject towards administration of an ErbB2 targeting treatment.

31. A kit for detecting the presence or absence of EGFR expression comprising: antibody to EGFR, antibody to tumor endothelial cell antigens, antibody to endothelial cell antigens or any combination thereof.

32. A method of screening for EGFR targeting agents, wherein an agent is screened for effectiveness targeting EGFR, wherein tumor epithelial cells expressing EGFR are administered the agent, wherein the response of the tumor epithelial cells to the agent is monitored.

33. The method of claim 32, wherein cessation of growth or death of the tumor endothelial cell indicates that the EGFR targeting agent is effective.

34. The method of claim 32, wherein reduction in EGFR phosphorylation indicates that the EGFR targeting agent is effective.

35. The method of claim 32, wherein reduction in EGFR expression indicates that the EGFR targeting agent is effective.

36. A method of screening for ErbB2 targeting agents, wherein an agent is screened for effectiveness targeting ErbB2, wherein tumor epithelial cells expressing ErbB2 are administered the agent, wherein the response of the tumor epithelial cells to the agent is monitored.

37. The method of claim 32, wherein cessation of growth or death of the tumor endothelial cell indicates that the ErbB2 targeting agent is effective.

38. The method of claim 32, wherein reduction in ErbB2 phosphorylation indicates that the ErbB2 targeting agent is effective.

39. The method of claim 32, wherein reduction in ErbB2 expression indicates that the ErbB2 targeting agent is effective.
